SEQUENCE LISTING (279 sequences)

SEQ ID NO: 1 - DNA, 18 bp, Escherichia coli
tgtgaattaatatgcaaa

SEQ ID NO: 2 - DNA, 18 bp, Escherichia coli
agtgagtgaatattctct

SEQ ID NO: 3 - DNA, 18 bp, Escherichia coli
tgtgatttttaattcaat

SEQ ID NO: 4 - DNA, 18 bp, Escherichia coli
aacgaatttaaattcatt

SEQ ID NO: 5 - DNA, 18 bp, Escherichia coli
attgaattaaaattcact

SEQ ID NO: 6 - DNA, 18 bp, Escherichia coli
tatgtgtaattattcatt

SEQ ID NO: 7 - DNA, 18 bp, Escherichia coli
tatgcttgatattgcact

SEQ ID NO: 8 - DNA, 18 bp, Escherichia coli
tttgcattttgattcaca

SEQ ID NO: 9 - DNA, 18 bp, Escherichia coli
tgtgatattttattgaat

SEQ ID NO: 10 - DNA, 18 bp, Escherichia coli
tttaaatattgtttttat

SEQ ID NO: 11 - DNA, 18 bp, Escherichia coli
acagaataattatgcaat

SEQ ID NO: 12 - DNA, 18 bp, Escherichia coli
tctgctttaaaagttaaa

SEQ ID NO: 13 - DNA, 18 bp, Escherichia coli
actggataatcatgcatt

SEQ ID NO: 14 - DNA, 18 bp, Escherichia coli
gatggattttcaggaata

SEQ ID NO: 15 - DNA, 18 bp, Escherichia coli
actccatttttatgcgtt

SEQ ID NO: 16 - DNA, 18 bp, Escherichia coli
ccctaatatttattcttc

SEQ ID NO: 17 - DNA, 18 bp, Escherichia coli
aatgcatacttaagcatt

SEQ ID NO: 18 - DNA, 18 bp, Escherichia coli
aatgaaaaataatacgtc

SEQ ID NO: 19 - DNA, 18 bp, Escherichia coli
aaggcataaaaagtcgat

SEQ ID NO: 20 - DNA, 18 bp, Escherichia coli
gttgaatattttttcagc

SEQ ID NO: 21 - DNA, 18 bp, Escherichia coli
tatttatattcatgcgtt

SEQ ID NO: 22 - DNA, 18 bp, Escherichia coli
tatgaaagtttatgcacc

SEQ ID NO: 23 - DNA, 18 bp, Escherichia coli
aatgagtatttattctca

SEQ ID NO: 24 - DNA, 18 bp, Escherichia coli
atcgaaaaaggattcatt

SEQ ID NO: 25 - DNA, 18 bp, Escherichia coli
acagaataaaaatacact

SEQ ID NO: 26 - DNA, 18 bp, Escherichia coli
ttcgaataatcatgcaaa

SEQ ID NO: 27 - DNA, 18 bp, Escherichia coli
attaaatgaaaactcatt

SEQ ID NO: 28 - DNA, 18 bp, Escherichia coli
tttgcataaaaattcagt

SEQ ID NO: 29 - DNA, 18 bp, Escherichia coli
actgcataatcaatcaaa

SEQ ID NO: 30 - DNA, 18 bp, Escherichia coli
accgaaatttcatgcata

SEQ ID NO: 31 - DNA, 18 bp, Escherichia coli
tttgcataaaaattcatc

SEQ ID NO: 32 - DNA, 18 bp, Escherichia coli
tatgcacaataatgttgt

SEQ ID NO: 33 - DNA, 18 bp, Escherichia coli
tgtgaaattataaccaca

SEQ ID NO: 34 - DNA, 18 bp, Escherichia coli
tatgcataaaaaatcact

SEQ ID NO: 35 - DNA, 18 bp, Escherichia coli
tatcaatattcatgcagt

SEQ ID NO: 36 - DNA, 18 bp, Escherichia coli
tatgaataaaaatacact

SEQ ID NO: 37 - DNA, 18 bp, Escherichia coli
aatgaataatcatccata

SEQ ID NO: 38 - DNA, 18 bp, Escherichia coli
attgaattttaattcatt

SEQ ID NO: 39 - DNA, 18 bp, Escherichia coli
agagaatagctatgcatg

SEQ ID NO: 40 - DNA, 18 bp, Escherichia coli
caggaataaccatgcaac

SEQ ID NO: 41 - DNA, 18 bp, Escherichia coli
tctggatggttatgtact

SEQ ID NO: 42 - DNA, 18 bp, Escherichia coli
ttttaatttttatgaggc

SEQ ID NO: 43 - DNA, 18 bp, Escherichia coli
gctgaatcgctattcatt

SEQ ID NO: 44 - DNA, 18 bp, Escherichia coli
tccgaaatttcatgcatg

SEQ ID NO: 45 - DNA, 18 bp, Escherichia coli
tatgaataaacacgcatc

SEQ ID NO: 46 - DNA, 18 bp, Escherichia coli
gtggaattattatgaata

SEQ ID NO: 47 - DNA, 18 bp, Escherichia coli
attgcataaataatcaga

SEQ ID NO: 48 - DNA, 18 bp, Escherichia coli
aatgtatatttatgcaat

SEQ ID NO: 49 - DNA, 18 bp, Escherichia coli
gcagcataaaaatgcaat

SEQ ID NO: 50 - DNA, 18 bp, Escherichia coli
tcggtattttcattcagc

SEQ ID NO: 51 - DNA, 18 bp, Escherichia coli
attgaatctttattagtt

SEQ ID NO: 52 - DNA, 18 bp, Escherichia coli
aaagcatatttattccga

SEQ ID NO: 53 - DNA, 18 bp, Escherichia coli
tatgcataaccatgcaga

SEQ ID NO: 54 - DNA, 18 bp, Escherichia coli
tctgcatggttatgcata

SEQ ID NO: 55 - DNA, 18 bp, Escherichia coli
attgcataaactttaaaa

SEQ ID NO: 56 - DNA, 18 bp, Escherichia coli
aacgtattttttttgaat

SEQ ID NO: 57 - DNA, 18 bp, Escherichia coli
tctgcatttttattcctt

SEQ ID NO: 58 - DNA, 18 bp, Escherichia coli
catggattttaatggatt

SEQ ID NO: 59 - DNA, 18 bp, Escherichia coli
tatgcataaaaatttgtt

SEQ ID NO: 60 - DNA, 18 bp, Escherichia coli
attgcatataaattcact

SEQ ID NO: 61 - DNA, 18 bp, Escherichia coli
agtgcataaattcttata

SEQ ID NO: 62 - DNA, 18 bp, Escherichia coli
tctgcattattctgccgt

SEQ ID NO: 63 - DNA, 18 bp, Escherichia coli
tatgcataacatttcctt

SEQ ID NO: 64 - DNA, 18 bp, Escherichia coli
catgcatggttatgcgca

SEQ ID NO: 65 - DNA, 18 bp, Escherichia coli
aatgaatgtctatccagt

SEQ ID NO: 66 - DNA, 18 bp, Escherichia coli
tatgcatatttatgggaa

SEQ ID NO: 67 - DNA, 18 bp, Escherichia coli
ggtgcaaatttattctgg

SEQ ID NO: 68 - DNA, 18 bp, Escherichia coli
gatgaataaatatgccca

SEQ ID NO: 69 - DNA, 18 bp, Escherichia coli
cttgcatgaagatgcagg

SEQ ID NO: 70 - DNA, 18 bp, Escherichia coli
tgcgaataaatatgcacc

SEQ ID NO: 71 - DNA, 18 bp, Escherichia coli
catgattatttatgatct

SEQ ID NO: 72 - DNA, 18 bp, Escherichia coli
gttgcatgattatttatg

SEQ ID NO: 73 - DNA, 18 bp, Escherichia coli
atcacatatttatgcact

SEQ ID NO: 74 - DNA, 18 bp, Escherichia coli
agtcaataaaattgccga

SEQ ID NO: 75 - DNA, 18 bp, Escherichia coli
tataaataactattcagt

SEQ ID NO: 76 - DNA, 18 bp, Escherichia coli
ttcgcatttttagtcaca

SEQ ID NO: 77 - DNA, 18 bp, Escherichia coli
cgcgaataacttagcaat

SEQ ID NO: 78 - DNA, 18 bp, Escherichia coli
attgttaatttcttcact

SEQ ID NO: 79 - DNA, 18 bp, Escherichia coli
tctgcataacattgcgtt

SEQ ID NO: 80 - DNA, 18 bp, Escherichia coli
agtgcaaaaatattcaga

SEQ ID NO: 81 - DNA, 18 bp, Escherichia coli
tttgcataaaaaatcagg

SEQ ID NO: 82 - DNA, 18 bp, Escherichia coli
aaccaatcactattcaca

SEQ ID NO: 83 - DNA, 18 bp, Escherichia coli
cgtgaataatattcaaca

SEQ ID NO: 84 - DNA, 18 bp, Escherichia coli
cctgattattcatgaagt

SEQ ID NO: 85 - DNA, 18 bp, Escherichia coli
tctgaatatttctttagc

SEQ ID NO: 86 - DNA, 18 bp, Escherichia coli
aaagaaaaattattctga

SEQ ID NO: 87 - DNA, 18 bp, Escherichia coli
gttgaataaacattcaca

SEQ ID NO: 88 - DNA, 18 bp, Escherichia coli
tacgcatagctatgcaga

SEQ ID NO: 89 - DNA, 18 bp, Escherichia coli
gtggcattaaaattaatc

SEQ ID NO: 90 - DNA, 18 bp, Escherichia coli
tcggtttgtttatgcatt

SEQ ID NO: 91 - DNA, 18 bp, Escherichia coli
tccggttttttattctct

SEQ ID NO: 92 - DNA, 18 bp, Escherichia coli
aacgcatcaatacgcaat

SEQ ID NO: 93 - DNA, 18 bp, Escherichia coli
ggtgcattgaaatgcata

SEQ ID NO: 94 - DNA, 18 bp, Escherichia coli
aatgaataattaagcggc

SEQ ID NO: 95 - DNA, 18 bp, Escherichia coli
tgcgaataataaaaaaaa

SEQ ID NO: 96 - DNA, 18 bp, Escherichia coli
gctgtaattttttttaac

SEQ ID NO: 97 - DNA, 18 bp, Escherichia coli
tgtttaatattatttaat

SEQ ID NO: 98 - DNA, 18 bp, Escherichia coli
acggcataattacgcatt

SEQ ID NO: 99 - DNA, 18 bp, Escherichia coli
ctaaactaaaaatgcaac

SEQ ID NO: 100 - DNA, 18 bp, Escherichia coli
attgcatttttattcact

SEQ ID NO: 101 - DNA, 18 bp, Escherichia coli
tatgatttatttttaatt

SEQ ID NO: 102 - DNA, 18 bp, Escherichia coli
aatttttatttagtcagt

SEQ ID NO: 103 - DNA, 18 bp, Escherichia coli
gaaaaatttttatttagt

SEQ ID NO: 104 - DNA, 18 bp, Escherichia coli
gttgtttattcatgcgtt

SEQ ID NO: 105 - DNA, 18 bp, Escherichia coli
gctgaataattattccca

SEQ ID NO: 106 - DNA, 18 bp, Escherichia coli
tctgttaatttttgctct

SEQ ID NO: 107 - DNA, 18 bp, Escherichia coli
tgtgaatgaatatccagt

SEQ ID NO: 108 - DNA, 18 bp, Escherichia coli
agcgcattattttacaaa

SEQ ID NO: 109 - DNA, 18 bp, Escherichia coli
ctcccataactattcatt

SEQ ID NO: 110 - DNA, 18 bp, Escherichia coli
tcatcatatttatgcagt

SEQ ID NO: 111 - DNA, 18 bp, Escherichia coli
cctgaataaagatcctga

SEQ ID NO: 112 - DNA, 18 bp, Escherichia coli
cgtgaaaaaatattcaca

SEQ ID NO: 113 - DNA, 18 bp, Escherichia coli
tatgcatattttgtggtt

SEQ ID NO: 114 - DNA, 18 bp, Escherichia coli
gatgatttatcattcagg

SEQ ID NO: 115 - DNA, 18 bp, Escherichia coli
atcgcataaaaatacaaa

SEQ ID NO: 116 - DNA, 18 bp, Escherichia coli
atacaaaaaaaatgaaga

SEQ ID NO: 117 - DNA, 18 bp, Escherichia coli
agtaaataaatatgctgt

SEQ ID NO: 118 - DNA, 18 bp, Escherichia coli
atatcataaacttgcaaa

SEQ ID NO: 119 - DNA, 18 bp, Escherichia coli
ggtaatttgtttttcatt

SEQ ID NO: 120 - DNA, 18 bp, Escherichia coli
gctgcataaaaaataatc

SEQ ID NO: 121 - DNA, 18 bp, Escherichia coli
atgccaaaaataatcact

SEQ ID NO: 122 - DNA, 18 bp, Escherichia coli
aacaaatggttattcatt

SEQ ID NO: 123 - DNA, 18 bp, Escherichia coli
ctcgcgttattatgcatt

SEQ ID NO: 124 - DNA, 18 bp, Escherichia coli
ggtgactaaaaatccagc

SEQ ID NO: 125 - DNA, 18 bp, Escherichia coli
atcaaataaaaatgcata

SEQ ID NO: 126 - DNA, 18 bp, Escherichia coli
tgcaaatgaataatcatc

SEQ ID NO: 127 - DNA, 15 bp, Escherichia coli
tgtaatttttatctg

SEQ ID NO: 128 - DNA, 15 bp, Escherichia coli
tagctaattgtgctg

SEQ ID NO: 129 - DNA, 15 bp, Escherichia coli
caaaaatttatgctg

SEQ ID NO: 130 - DNA, 15 bp, Escherichia coli
ctgcattttttattg

SEQ ID NO: 131 - DNA, 15 bp, Escherichia coli
tagaattttattctg

SEQ ID NO: 132 - DNA, 15 bp, Escherichia coli
cagaaatttctgcta

SEQ ID NO: 133 - DNA, 15 bp, Escherichia coli
cagaaatttctgctc

SEQ ID NO: 134 - DNA, 15 bp, Escherichia coli
gagcattttctgcta

SEQ ID NO: 135 - DNA, 15 bp, Escherichia coli
cggtttttgctgctt

SEQ ID NO: 136 - DNA, 15 bp, Escherichia coli
tgccggtttttgctg

SEQ ID NO: 137 - DNA, 15 bp, Escherichia coli
caacattttctgcta

SEQ ID NO: 138 - DNA, 15 bp, Escherichia coli
cgatagtgtttgctg

SEQ ID NO: 139 - DNA, 15 bp, Escherichia coli
ttgcattttatattg

SEQ ID NO: 140 - DNA, 15 bp, Escherichia coli
taaaatatcttactg

SEQ ID NO: 141 - DNA, 15 bp, Escherichia coli
caacatttccttctg

SEQ ID NO: 142 - DNA, 15 bp, Escherichia coli
cagatgttttcccgg

SEQ ID NO: 143 - DNA, 15 bp, Escherichia coli
cagatgattccactg

SEQ ID NO: 144 - DNA, 15 bp, Escherichia coli
caatgttttttgccg

SEQ ID NO: 145 - DNA, 15 bp, Escherichia coli
tgtctgttttttctg

SEQ ID NO: 146 - DNA, 15 bp, Escherichia coli
tagtgttttatactg

SEQ ID NO: 147 - DNA, 15 bp, Escherichia coli
taaatttttgtgcca

SEQ ID NO: 148 - DNA, 15 bp, Escherichia coli
cgaaatattatgcta

SEQ ID NO: 149 - DNA, 15 bp, Escherichia coli
cagatgattaccctg

SEQ ID NO: 150 - DNA, 15 bp, Escherichia coli
ccgaatttcatcctg

SEQ ID NO: 151 - DNA, 15 bp, Escherichia coli
ccgctttttctgctg

SEQ ID NO: 152 - DNA, 15 bp, Escherichia coli
cggctttttctgccg

SEQ ID NO: 153 - DNA, 15 bp, Escherichia coli
cagcttttgccgctg

SEQ ID NO: 154 - DNA, 15 bp, Escherichia coli
cggctttttcagctg

SEQ ID NO: 155 - DNA, 15 bp, Escherichia coli
tggcagattatgttg

SEQ ID NO: 156 - DNA, 15 bp, Escherichia coli
cggaatttcaccccg

SEQ ID NO: 157 - DNA, 15 bp, Escherichia coli
cggtggttttttcta

SEQ ID NO: 158 - DNA, 15 bp, Escherichia coli
cagattatcatcctg

SEQ ID NO: 159 - DNA, 15 bp, Escherichia coli
caaaattatctgctg

SEQ ID NO: 160 - DNA, 15 bp, Escherichia coli
cgttattttgtgcta

SEQ ID NO: 161 - DNA, 15 bp, Escherichia coli
caactgtttttcctg

SEQ ID NO: 162 - DNA, 15 bp, Escherichia coli
ccgatgtttttactg

SEQ ID NO: 163 - DNA, 15 bp, Escherichia coli
caatttattatgctg

SEQ ID NO: 164 - DNA, 15 bp, Escherichia coli
cagttttttctgctg

SEQ ID NO: 165 - DNA, 15 bp, Escherichia coli
gagaatttttttctt

SEQ ID NO: 166 - DNA, 15 bp, Escherichia coli
cggaattttatgctg

SEQ ID NO: 167 - DNA, 15 bp, Escherichia coli
taatattttcaactg

SEQ ID NO: 168 - DNA, 15 bp, Escherichia coli
tagattattattctt

SEQ ID NO: 169 - DNA, 15 bp, Escherichia coli
cggatcatttcgctg

SEQ ID NO: 170 - DNA, 15 bp, Escherichia coli
aaacatattgtgctg

SEQ ID NO: 171 - DNA, 15 bp, Escherichia coli
cggtattgtgtgctc

SEQ ID NO: 172 - DNA, 15 bp, Escherichia coli
cgaatgattttgttg

SEQ ID NO: 173 - DNA, 15 bp, Escherichia coli
cagcattttatgttg

SEQ ID NO: 174 - DNA, 15 bp, Escherichia coli
cattatttttcgctg

SEQ ID NO: 175 - DNA, 15 bp, Escherichia coli
cggcatttttcttcg

SEQ ID NO: 176 - DNA, 15 bp, Escherichia coli
tggctgttcctgctg

SEQ ID NO: 177 - DNA, 15 bp, Escherichia coli
ccgcagattgtgctg

SEQ ID NO: 178 - DNA, 15 bp, Escherichia coli
cggcgctttttgcta

SEQ ID NO: 179 - DNA, 15 bp, Escherichia coli
cagaatatttatctg

SEQ ID NO: 180 - DNA, 15 bp, Escherichia coli
cggcagttgatgctg

SEQ ID NO: 181 - DNA, 15 bp, Escherichia coli
tggtgatttttgctg

SEQ ID NO: 182 - DNA, 15 bp, Escherichia coli
taattttttctgccg

SEQ ID NO: 183 - DNA, 15 bp, Escherichia coli
gagaatttgatgctg

SEQ ID NO: 184 - DNA, 15 bp, Escherichia coli
tgatattttctgctt

SEQ ID NO: 185 - DNA, 15 bp, Escherichia coli
cggaagatcgtgctg

SEQ ID NO: 186 - DNA, 15 bp, Escherichia coli
cagattttttcactg

SEQ ID NO: 187 - DNA, 15 bp, Escherichia coli
ccgaatttttcactg

SEQ ID NO: 188 - DNA, 15 bp, Escherichia coli
cagtattttgtatgg

SEQ ID NO: 189 - DNA, 15 bp, Escherichia coli
cagatgattattctg

SEQ ID NO: 190 - DNA, 15 bp, Escherichia coli
tgatattttgcgctt

SEQ ID NO: 191 - DNA, 15 bp, Escherichia coli
cggtagattatcctc

SEQ ID NO: 192 - DNA, 15 bp, Escherichia coli
caaaattatatgctg

SEQ ID NO: 193 - DNA, 15 bp, Escherichia coli
gagatatttctgctg

SEQ ID NO: 194 - DNA, 15 bp, Escherichia coli
cagtattgtttgccg

SEQ ID NO: 195 - DNA, 15 bp, Escherichia coli
cgtaatattatgccg

SEQ ID NO: 196 - DNA, 15 bp, Escherichia coli
gaacatttgttgctg

SEQ ID NO: 197 - DNA, 15 bp, Escherichia coli
tggtttttttatctg

SEQ ID NO: 198 - DNA, 15 bp, Escherichia coli
cggttttttattctc

SEQ ID NO: 199 - DNA, 15 bp, Escherichia coli
cggtagaatattctg

SEQ ID NO: 200 - DNA, 15 bp, Escherichia coli
tagaatattctgcta

SEQ ID NO: 201 - DNA, 15 bp, Escherichia coli
cagttgttaattctg

SEQ ID NO: 202 - DNA, 15 bp, Escherichia coli
cggcattttcttcgc

SEQ ID NO: 203 - DNA, 15 bp, Escherichia coli
cgattttttttgtgg

SEQ ID NO: 204 - DNA, 15 bp, Escherichia coli
tacaacttttcgctg

SEQ ID NO: 205 - DNA, 15 bp, Escherichia coli
cagtttattgagctg

SEQ ID NO: 206 - DNA, 15 bp, Escherichia coli
cagaatattttgctg

SEQ ID NO: 207 - DNA, 15 bp, Escherichia coli
tggtgctttctgctg

SEQ ID NO: 208 - DNA, 15 bp, Escherichia coli
cgctattttctgctg

SEQ ID NO: 209 - DNA, 15 bp, Escherichia coli
tgtatttttattctg

SEQ ID NO: 210 - DNA, 15 bp, Escherichia coli
tggttttttattctg

SEQ ID NO: 211 - DNA, 15 bp, Escherichia coli
tggatgaatatgctg

SEQ ID NO: 212 - DNA, 15 bp, Escherichia coli
tggaaatttttcctg

SEQ ID NO: 213 - DNA, 15 bp, Escherichia coli
tacttttttatgccg

SEQ ID NO: 214 - DNA, 15 bp, Escherichia coli
tggcatttgctgctg

SEQ ID NO: 215 - DNA, 15 bp, Escherichia coli
caatatttaatgcta

SEQ ID NO: 216 - DNA, 15 bp, Escherichia coli
cagcatttttcgctt

SEQ ID NO: 217 - DNA, 15 bp, Escherichia coli
ctgatttttatactt

SEQ ID NO: 218 - DNA, 15 bp, Escherichia coli
tagcattttgtgttg

SEQ ID NO: 219 - DNA, 15 bp, Escherichia coli
gggatgttttctctg

SEQ ID NO: 220 - DNA, 15 bp, Escherichia coli
cactatttttcactg

SEQ ID NO: 221 - DNA, 15 bp, Escherichia coli
cagcattttatactg

SEQ ID NO: 222 - DNA, 15 bp, Escherichia coli
tagtattttatccgg

SEQ ID NO: 223 - DNA, 15 bp, Escherichia coli
aaaatttttatgctg

SEQ ID NO: 224 - DNA, 15 bp, Escherichia coli
cgttattttatgctg

SEQ ID NO: 225 - DNA, 15 bp, Escherichia coli
taaattatttcgcta

SEQ ID NO: 226 - DNA, 15 bp, Escherichia coli
tggcgtttcatgctg

SEQ ID NO: 227 - DNA, 15 bp, Escherichia coli
tagcagagtatgctg

SEQ ID NO: 228 - DNA, 15 bp, Escherichia coli
tggtttatcgtgctg

SEQ ID NO: 229 - DNA, 15 bp, Escherichia coli
gggattattaagctg

SEQ ID NO: 230 - DNA, 15 bp, Escherichia coli
gagagtattctgctg

SEQ ID NO: 231 - DNA, 15 bp, Escherichia coli
cgatattttttgctg

SEQ ID NO: 232 - DNA, 15 bp, Escherichia coli
tggcagtttttgcgc

SEQ ID NO: 233 - DNA, 15 bp, Escherichia coli
tggcactttattccg

SEQ ID NO: 234 - DNA, 15 bp, Escherichia coli
cagcatttggttccg

SEQ ID NO: 235 - DNA, 15 bp, Escherichia coli
tggatttatatgctg

SEQ ID NO: 236 - DNA, 15 bp, Escherichia coli
ctaaatattaagctg

SEQ ID NO: 237 - DNA, 15 bp, Escherichia coli
tggcattttccgctg

SEQ ID NO: 238 - DNA, 15 bp, Escherichia coli
tgacattttatcctg

SEQ ID NO: 239 - DNA, 15 bp, Escherichia coli
cggtgctttatcctg

SEQ ID NO: 240 - DNA, 15 bp, Escherichia coli
cagaatattgcacta

SEQ ID NO: 241 - DNA, 15 bp, Escherichia coli
tgattgattttactg

SEQ ID NO: 242 - DNA, 15 bp, Escherichia coli
tggcgttttgtgctg

SEQ ID NO: 243 - DNA, 15 bp, Escherichia coli
cagcgtatctcgctg

SEQ ID NO: 244 - DNA, 15 bp, Escherichia coli
tagatcattttgccg

SEQ ID NO: 245 - DNA, 15 bp, Escherichia coli
caacatttattgccg

SEQ ID NO: 246 - DNA, 15 bp, Escherichia coli
tccatttttatgctg

SEQ ID NO: 247 - DNA, 15 bp, Escherichia coli
tggttctttgtgctg

SEQ ID NO: 248 - DNA, 15 bp, Escherichia coli
caacattttctgctg

SEQ ID NO: 249 - DNA, 15 bp, Escherichia coli
caattgttaatgctg

SEQ ID NO: 250 - DNA, 15 bp, Escherichia coli
cagaattttgtgctg

SEQ ID NO: 251 - DNA, 15 bp, Escherichia coli
cgaaatattatgctg

SEQ ID NO: 252 - DNA, 15 bp, Escherichia coli
cggatcattgtgcgg

SEQ ID NO: 253 - DNA, 15 bp, Escherichia coli
cggtgtattttgcgg

SEQ ID NO: 254 - DNA, 15 bp, Escherichia coli
cagaatttttaaccg

SEQ ID NO: 255 - DNA, 15 bp, Escherichia coli
cagagcttttcgctg

SEQ ID NO: 256 - DNA, 15 bp, Escherichia coli
gggatttatatgctg

SEQ ID NO: 257 - DNA, 15 bp, Escherichia coli
cagtattttttactc

SEQ ID NO: 258 - DNA, 15 bp, Escherichia coli
cgctattttgtgttg

SEQ ID NO: 259 - DNA, 15 bp, Escherichia coli
tagatgtttatattg

SEQ ID NO: 260 - DNA, 15 bp, Escherichia coli
ttaattttcatgctg

SEQ ID NO: 261 - DNA, 15 bp, Escherichia coli
tggaatttgatgttg

SEQ ID NO: 262 - DNA, 15 bp, Escherichia coli
tggctgattatgcgg

SEQ ID NO: 263 - DNA, 15 bp, Escherichia coli
cagctgtttttgctt

SEQ ID NO: 264 - DNA, 15 bp, Escherichia coli
cggtatttaacgccg

SEQ ID NO: 265 - DNA, 15 bp, Escherichia coli
cgactttttgccctg

SEQ ID NO: 266 - DNA, 8 bp, Escherichia coli
tcacaacg

SEQ ID NO: 267 - DNA, 8 bp, Escherichia coli
tcacaacg

SEQ ID NO: 268 - DNA, 8 bp, Escherichia coli
tcaccatg

SEQ ID NO: 269 - DNA, 8 bp, Escherichia coli
tcaccatg

SEQ ID NO: 270 - DNA, 8 bp, Escherichia coli
tcacgatg

SEQ ID NO: 271 - DNA, 8 bp, Escherichia coli
tcaccacg

SEQ ID NO: 272 - DNA, 8 bp, Escherichia coli
tcacgacg

SEQ ID NO: 273 - DNA, 8 bp, Escherichia coli
tcacgacg

SEQ ID NO: 274 - DNA, 8 bp, Escherichia coli
tcaccacg

SEQ ID NO: 275 - DNA, 8 bp, Escherichia coli
tcacgacg

SEQ ID NO: 276 - DNA, 8 bp, Escherichia coli
tcacgacg

SEQ ID NO: 277 - DNA, 8 bp, Escherichia coli
tcacgacg

SEQ ID NO: 278 - DNA, 8 bp, Escherichia coli
tcaccacg

SEQ ID NO: 279 - DNA, 8 bp, Escherichia coli
tcaccacg

(19) United States
(12) Patent Application Publication
(10) Pub. No.: US 2015/0011400 A1
(43) Pub. Date: Jan. 8, 2015

(54) Bacterial Metastructure and Methods of Use

(71) Applicant: The Regents of the University of California, Oakland, CA (US)

(72) Inventors: Bernhard Palsson, San Diego, CA (US); Byung-kwan Cho, Daejeon (KR)

(21) Appl. No.: 14/356,588

(22) PCT Filed: Nov. 9, 2012
(86) PCT No.: PCT/US2012/064516

§ 371 Date: May 6, 2014

Related U.S. Application Data
(60) Provisional application No. 61/558,331, filed on Nov. 10, 2011.

(51) Int. Cl. G06F 19/12; C12N 1/20; G06F 19/22; G06F 19/18

(52) U.S. Cl. CPC G06F 19/12; G06F 19/18; C12N 1/20; G06F 19/22
USPC 506/2; 536/24.1; 435/375

(57) ABSTRACT

Although metabolic networks have been reconstructed on a genome-scale, the corresponding reconstruction and integration of governing transcriptional regulatory networks has not been fully achieved. Here such an integrated network was constructed for amino acid metabolism in Escherichia coli. Analysis of ChlP-chip and gene expression data for the transcription factors ArgR, Lrp, and TrpR showed that 19/20 amino acid biosynthetic pathways are either directly or indirectly controlled by these regulators. Classifying the regulated genes into three functional categories of transport, biosynthesis, and metabolism leads to elucidation of regulatory motifs constituting the integrated network's basic building blocks. The regulatory logic of these motifs was determined based on the relationships between transcription factor binding and changes in transcript levels in response to exogenous amino acids. Remarkably, the resulting logic shows how amino acids are differentiated as signaling and nutrient molecules. This reveals the overarching regulatory principles of the amino acid stimulon.

### FIELD OF THE INVENTION

The invention relates generally to determining the regulatory mechanisms for amino acid metabolism in bacterial genomes, and more specifically to methods for iteratively integrating multiple genome-scale measurements on the basis of genetic information flow to identify regulatory motifs for amino acid metabolism.

### BACKGROUND OF THE INVENTION

Transcriptional regulatory networks (TRN) in bacteria govern metabolic flexibility and robustness in response to environmental signals. Thus, causal relationships between transcript levels for metabolic genes and the direct association of transcription factors (TFs) at the genome-scale is fundamental to fully understand bacterial responses to their environment. In particular, the molecular interaction between small molecules ranging from nutrients to trace elements and TFs governs the TRN and ultimately regulates the related metabolic pathways. From the causal relationships, a small set of recurring regulation patterns, or regulatory motifs are identified and reconstructed to describe the design principles of complex biological systems. One primary discovery from this effort is the connected feedback circuit which coordinates influx (biosynthesis and transport) and efflux (metabolism) pathways that are jointly regulated by a TF sensing the relevant small molecule. For example, a part of the global TRN is comprised of certain TFs (ArgR, Lrp, and TrpR) that sense the presence of exogenous amino acids (arginine, leucine, and tryptophan, respectively) and, in response, regulate the expression of a number of target genes. Upon addition of these amino acids to the environment, the TFs exhibit enhanced, reversed, or unaffected regulatory modes. These TF responses make these amino acids not just nutrients but also signaling molecules.

Previously discovered regulatory motifs represent a significant step forward in the understanding of complex biological behavior. However, they fall short for appropriately elucidating the system wide response since they were either based upon incomplete information, or were only specific to a single transcription factor and regulon. This shortcoming has resulted in an inability to appropriately understand complex regulatory phenomena existing across multiple TFs and regulatory signals. Hence, it is necessary to achieve a full elucidation of these interactions with systematic and integrated experimental analysis.

Comprehensive elucidation of the causal relationships between TFs and genes is achievable by integrated analysis of expression data obtained from microarray or sequencing with direct TF-binding information from chromatin immunoprecipitation coupled with microarrays or sequencing (ChIP-chip or ChIP-seq). Here a genome-scale expression profiling and ChIP-chip for each TF to reconstruct regulons involved in amino acid metabolism at the genome-scale was obtained and integrated. The elucidated regulatory logic fell into two categories that differentiate the role of amino acids as signaling and as nutrient molecules. Therefore, the reconstruction of the regulatory logic of the regulatory motif allowed us to establish the physiological role of each TF regulon and to determine how they govern amino acid regulation in E. coli. The integration of these multiple regulons into a unified network led to the first full bottom-up genome-scale reconstruction of a stimulon.

Establishing the regulatory motif for amino acid metabolism is a challenging task. In-depth analyses of the transcriptomes and proteomes of multiple prokaryotic organisms indicate that the information content and structure of a genome is much more complex than previously thought, and that the process of revealing the role of cellular components in transcription and translation on a genome scale has just begun.

### SUMMARY OF THE INVENTION

The present invention is based on the finding that multiple genome-scale measurements may be used to determine the regulatory mechanisms for amino acid metabolism in bacterial genomes. As such, the invention provides a method that iteratively integrates multiple genome-scale measurements on the basis of genetic information flow to identify the regulatory motifs associated with amino acid metabolism.

In one embodiment, the present invention provides a method of identifying a regulatory motif for amino acid metabolism in a target organism comprising (a) obtaining the full genome sequence a target organism; (b) obtaining the genome-wide binding of a transcription factor from the organism; (c) obtaining the sequence of the binding sites from the organism; (d) obtaining the data described in (b) and (c) under a series of culture conditions for the organism; and (e) iteratively mapping the data sets described in (d) onto the DNA sequence in (a) and identify binding sites associated with genes involved in amino acid metabolism, thereby identifying the regulatory motif for amino acid metabolism in the target organism.

In one aspect, the target organism is a bacterial organism. For example, the target organism may be E. coli. In an additional aspect, the genome-wide binding of the transcription factor is obtained by chromatin immunoprecipitation coupled with a microarray. In a further aspect, the genome-wide binding of the transcription factor is obtained by deep sequencing of immunoprecipitated DNA. In an aspect, the sequence of the binding sites is obtained using tiled expression arrays. In a further aspect, the sequence of the binding sites is obtained using deep sequencing of the isolated DNA. In an additional aspect, the regulatory motif is associated with amino acid transport, biosynthesis or utilization. Further, the transcription factor may be ArgR, Lrp, TrpR, TyrR, PurR, PyrR, Fnr, ArcA, Crp, Cra, DgsA, Fis, Hns, HU, Ihf, StpA and Dps. In one aspect, one or more small molecules is used to produce the series of culture conditions. Further, the small molecule may be an amino acid.

In an additional embodiment, the present invention discloses a regulatory motif for ArpR binding wherein the motif is selected from the group consisting of SEQ ID NOs:1-126. In another embodiment, the present invention is a regulatory motif for Lrp binding selected from the group consisting of SEQ ID NOs:127-265. In a further embodiment, the present invention is a regulatory motif for TrpR binding selected from the group consisting of SEQ ID NOs:266-279.

One embodiment of the present invention discloses a method of modulating ArgR activity comprising contacting ArgR with a small molecule. In one aspect, the small molecule is an amino acid. Further, the amino acid may be phenylalanine, tyrosine, tryptophan, lysine, arginine, histidine, aspartic acid, glutamic acid, valine, isoleucine, leucine, alanine, glycine, serine, threonine and proline. In an aspect, the modulated activity is activation or repression of at least one pathway. Further, the pathway maybe an amino acid transportation, biosynthesis or utilization pathway. In one aspect, the amino acid is phenylalanine, tyrosine or tryptophan and the modulated activity is activation of transportation or utilization pathway and repression of a biosynthesis pathway. In an additional aspect, the amino acid is lysine, arginine or histidine and the modulated activity is activation of a utilization pathway and repression of a biosynthesis and a transportation pathway. In a further aspect, the amino acid is asparagine or glutamine and the modulated activity is activation of transportation pathway and repression of a utilization and a biosynthesis pathway.

In one embodiment, the present invention is a method of modulating Lrp activity comprising contacting Lrp with a small molecule. In one aspect, the small molecule is an amino acid. In one aspect, the amino acid is an essential amino acid, such as phenylalanine, tyrosine, tryptophan, lysine, arginine, histidine, aspartic acid, glutamic acid, valine, isoleucine, leucine, alanine, glycine, serine, threonine and proline. In an aspect, the modulated activity is activation or repression of a at least one pathway. Further, the pathway maybe an amino acid transportation, biosynthesis or utilization pathway. In an aspect, the modulated activity is activation or repression of at least one pathway. Further, the pathway maybe an amino acid transportation, biosynthesis or utilization pathway. In one aspect, the amino acid is phenylalanine, tyrosine or tryptophan and the modulated activity is activation of transportation or utilization pathway and repression of a biosynthesis pathway. In another aspect, the amino acid is lysine, arginine or histidine and the modulated activity is activation of a transportation or utilization pathway and repression of a biosynthesis pathway. In a further aspect, the amino acid is asparagine or glutamic acid and the modulated activity is activation of a utilization or a biosynthesis pathway and repression of a transportation pathway. In yet another aspect, the amino acid is valine, isoleucine or leucine and the modulated activity is activation of a transportation or a biosynthesis pathway and repression of a utilization pathway. In an aspect, the amino acid is alanine, glycine, serine, threonine or proline and the modulated activity is activation of a transportation or a utilization or a pathway and repression of a biosynthesis pathway.

In one embodiment, the present invention provides an amino acid regulatory motif comprising the activation of a transportation and biosynthesis pathway and repression of a utilization pathway. In an additional aspect, the present invention is an amino acid regulatory motif which can be used in a method for the activation of a biosynthesis pathway and repression of a biosynthesis and a utilization pathway. In a further embodiment, the present invention is an amino acid regulatory motif which can be used in a method for the activation of a biosynthesis and utilization pathway and repression of a transportation pathway.

In one embodiment the present invention includes a method for modulating amino acid metabolism.

### BRIEF DESCRIPTION OF THE DRAWINGS

FIG. 1 depicts the genome-wide distribution of ArgR- and TrpR-binding regions (regulatory code analysis). (a) An overview of ArgR- and TrpR-binding profiles across the E. coli genome in the presence of exogenous arginine (upper track) and tryptophan (lower track). Enrichment fold on the y-axis was calculated from Cy5 (IP-DNA) and Cy3 (mock control) signal intensity of each probe and plotted against each location on the 4.64 Mb E. coli genome. Dots indicate the binding regions previously identified (filled) and newly determined (open). (b) Examples of genuine ArgR- (upper track), TrpR- (middle track), and Lrp-L-binding (lower track) regions on the selected regions. The gltBD operon is regulated by both ArgR and Lrp, whereas the aroH gene is solely regulated by TrpR. In the case of mtr, both TrpR and Lrp regulate its transcription. Both ArgR and Lrp regulate the potFGHI and artPIQM operons, however ArgR only regulates artJ. (c) Overlaps between ArgR-, Lrp-, and TrpR-binding regions.

FIG. 2 is a chart depicting the functional classification of genes directly regulated by ArgR, Lrp, and TrpR. The functions of regulon members are strongly enriched into amino acid, carbohydrate, membrane transport (mostly amino acid related), and energy metabolism.

FIG. 3 shows the delineation of amino acid biosynthetic pathways and transport systems in E. coli (topological analysis). (a) The amino acid biosynthetic pathways. The genes are directly regulated by ArgR, Lrp, and TrpR, respectively. The orange colored genes (gltB and gltD in glutamate biosynthesis, and argA in arginine biosynthesis) are regulated by both ArgR and Lrp. Green dots indicate the biosynthetic reactions, which use glutamate as an amino donor. (b) The amino acid transport systems. The genes encoding each transport system can be classified into ten groups (A˜J) based on the amino acid specificity. The transport systems in green color are directly regulated by ArgR, Lrp, or TrpR. For others, the transcriptional regulation has not been determined. FIG. 4 shows determination of TSS by mapping TSS reads to RTS, using a window size of 200 by and cutoff of 60%.

FIG. 4 shows the causal relationships between direct associations of transcription factors and the changes in RNA transcript levels of genes (functional analysis). Regulated genes are broken down into three main categories of transport, biosynthesis, or utilization of respective amino acids. They are further broken down based upon their amino acid specificities and pathway participation. Here tnaA was included as an indirectly regulated gene with significant differential expression but no ChIP enrichment in order to fully capture the utilization response to arginine. For class C it is noted that the direct targets of utilization for glutamate are in fact the biosynthetic genes that are shown in that section and pointed out in FIG. 3.

FIG. 5 depicts the reconstruction of regulatory motif and the logical structures of connected circuit motifs in response to the exogenous amino acids (network analysis). (a) Schematic diagram for the regulatory motif reconstruction in feedback circuit. (b) Logical structures of the regulatory motif in response to the exogenous amino acids. (c) The classification of function of amino acids derived from the logical structures of the connected feedback circuit motif. The utilization patterns for glutamate and aspartate are highly complex. Here it was concluded that the overall trend is repression given the role of glutamate as a substrate for nine biosynthetic pathways (FIG. 3, 4).

FIG. 6 shows the transcriptional co-regulation by ArgR and Lrp. (a) ArgR binding (top) and TrpR-binding (middle) profiles across the E. coli genome. Enrichment fold on the y-axis was calculated from Cy5 (IP-DNA) and Cy3 (mock control) signal intensity of each probe and plotted against each location on the 4.64 Mb E. coli genome. Black dotted box indicates the Lrp-L gene. (b) ArgR-binding (top) and TrpR-binding (middle) profiles at the argA gene (black dotted box). (c) ArgR-binding (top) and TrpR-binding (middle) profiles at the astCADBE operon (black dotted box). (d) ArgR-binding (top) and TrpR-binding (middle) profiles at the stpA gene (black dotted box). (e) ArgR-binding (top) and TrpR-binding (middle) profiles at the gltP gene (black dotted box).

FIG. 7 shows the sequences of ArgR-, Lrp-, and TrpR-binding regions. Using the MEME suite tool, the sequences of ArgR-, Lrp-, and TrpR-binding regions were used to generate the PSPM (position specific probability matrix) and to rescan the entire genome with the FIMO program. Only those sites were analyzed, which were located in the ArgR-, Lrp-, and TrpR-binding regions and fell below a stringent cut-off (P-value less than 10⁻⁴). This revealed a total of 124, 187, and 24 conserved sequences spread across 63, 141, and 8 binding regions, respectively (FIGS. 6a-c and 10-12). The data was consistent with the fact that a single ArgR-arginine complex hexamer binds to two partially conserved 18 bp-long imperfect palindromes (ARG boxes) separated by 2-3 bps, which overlapped with the core promoter elements, i.e., the Pribnow box and the transcription start site (TSS). In case of TrpR, the optimal half-site sequence for recognition by one TrpR dimer has been well documented and can be paired to form a palindrome structure around the core promoter elements. In the case of Lrp, it has been previously identified a 15-bp conserved sequence structured with flanking CAG/CTG triplets and a central AT-rich signal that together are reminiscent of DNA sequence characteristics important for nucleosome positioning and stability.

FIG. 8 is a table listing the ArgR associated regions identified by ChIP-chip analysis and its regulatory effect on the target operons determined by expression profiles. The table summarizes the results of ChIP-chip experiments to determine the genome-wide locations of DNA targets for ArgR binding in exponential phase E. coli cells growing in minimal medium in the presence (Arginine) and the absence (NH₄Cl) of arginine. First and second columns indicate the information of identified TrpR binding peaks (Start: left-end peak position, End: right end peak position). Third and fourth columns (Occupancy) indicate the log 2 ratio of each ArgR binding peaks.

FIG. 9 is a table listing the TrpR associated regions identified by ChIP-chip analysis and its regulatory effect on the target operons determined by expression profiles. The table summarizes the results of ChIP-chip experiments to determine the genome-wide locations of DNA targets for TrpR binding in exponential phase E. coli cells growing in minimal medium in the presence (Tryptophan) and the absence (NH₄Cl) of tryptophan. First and second columns indicate the information of identified ArgR binding peaks (Start: left-end peak position, End: right end peak position). Third and fourth columns (Occupancy) indicate the log 2 ratio of each TrpR binding peaks.

FIG. 10 is a table listing the motifs found confirming pattern of consecutive ArgR boxes (SEQ ID NOs: 1-126).

FIG. 11 is a table listing the motifs found confirming pattern of consecutive Lrp binding regions (SEQ ID NOs:127-265).

FIG. 12 is a table listing the motifs found confirming pattern TryR binding regions (SEQ ID NOs:266-279).

### DETAILED DESCRIPTION OF THE INVENTION

The present invention is based on the finding that multiple genome-scale measurements may be used to determine the regulatory mechanisms for amino acid metabolism in bacterial genomes. As such, the invention provides a method that iteratively integrates multiple genome-scale measurements on the basis of genetic information flow to identify the regulatory motifs associated with amino acid metabolism.

Before the present methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, the term “genome” refers to the entirety of an organism's hereditary information. It is encoded either in DNA or, for many types of virus, in RNA. The genome includes both the genes and the non-coding sequences of the DNA. Thus, a “gene” refers to a stretch of DNA that encodes for a functional polypeptide chain or RNA molecule. A gene is limited by a start codon and a stop codon. A codon is a sequence of three adjacent nucleotides in a nucleic acid that code for a specific amino acid. As used herein, the term “genetic” refers to the heritable information encoded in the sequence of DNA nucleotides. As such, the term “genetic characterization” is intended to mean the sequencing, genotyping, comparison, mapping or other assay of the information encoded in DNA. The scope (e.g., extent, scale, etc.) of the genetic characterization is substantially genomic in scale so that a comprehensive assessment of all the genetic elements (known or unknown) can be simultaneously assessed. Substantially comprehensive evaluation ideally includes a full genome-scale re-sequencing of the organism's genome. In cases where full genomic sequencing is not possible, such as due to extensive sequence repeat regions, a comprehensive draft of the genome sequence can be used in the method described.

As used herein, the term “genetic basis” refers to the underlying genetic or genomic cause of a particular observation. Also included in the term is the most important reason for the occurrence of the observation.

A “discrete genomic region” as used herein, is intended to mean a contiguous region or portion of a genome. A genome, or portion thereof, may be fractionated into any number of different discrete genomic regions to be analyzed. In one aspect, a discrete genomic region may be defined as a region of the genome including one or more probe sequences. In another aspect, a discrete genomic region may be defined as a region of the genome that includes two or more probe sequences separated by less than about 10,000, 5,000, 4,000, 3,000, 2,000 or 1,000 base pairs. “Tiling” refers to a process involving analyzing a particular discrete genomic region by moving along the genomic sequence in a frame-wise fashion to determine appropriate probe sequences used to generate probes that are used to manufacture the array. In various aspects, a genomic region may be tiled with different sizes of oligonucleotide sequences. For example, oligonucleotide sequences may be about 15-20, 20-25, 25-30, 30-35, 35-40, 40-45, 45-50, 50-55, 55-60, 60-65, 65-70, 70-75, 75-80, 80-85, 85-90, 90-95 or 95-100 base pairs in length. Additionally, the size of each frame may be determined by the length of the oligonucleotide used to tile the region and the frame of the frame-wise shift may overlap or skip regions of the genomic region by a specific number of base pairs. As such, in various aspects, about 1-25, 25-50, 50-75, 75-100 or more than 100 base pairs may be skipped in the tiling process to determine probe sequences within a region. In an exemplary aspect, tiling of the genomic region is performed using oligonucleotide sequences of about 50 base pairs and about 35 base pairs apart.

As used herein, the term “DNA” or “deoxyribonucleic acid” refers to a nucleic acid that contains the genetic instructions used in the development and functioning of all known living organisms. The main role of DNA molecules is the long-term storage of information.

As used herein, the term “5′-end” designates the end of the DNA or RNA strand that has the fifth carbon in the sugar-ring of the deoxyribose or ribose at its terminus.

The genomes of complex organisms are known to vary in GC content along their length. That is, they vary in the local proportion of the nucleotides G and C, as opposed to the nucleotides A and T. Changes in GC content are often abrupt, producing well-defined regions. Such abrupt changes are referred to herein as “change points”.

As used herein, a “transcription unit” (TU) refers to a stretch of DNA, which consists of a promoter site, 5′ untranslated (5′-UTR) sequence, a transcription terminator, 3′ untranslated (3′-UTR) sequence, and the stretch of DNA, which can be transcribed into an RNA molecule (can be mRNA, tRNA, rRNA, miscellaneous RNA). A gene or operon can be controlled by different promoters, hence, resulting in different TUs. Also, the operon length may vary depending on the transcriptional termination signal, yielding in different TUs.

As used herein, a “transcription start site” (TSS) refers to the genomic position where transcription begins. Primer extension can be used to determine the start site of RNA transcription for a known gene. This technique requires a radiolabelled primer (usually 20-50 nucleotides in length) which is complementary to a region near the 5′ end of the gene. The primer is allowed to anneal to the RNA and reverse transcriptase is used to synthesize complementary cDNA to the RNA until it reaches the 5′ end of the RNA. By running the product on a polyacrylamide gel, it is possible to determine the TSS, as the length of the sequence on the gel represents the distance from the start site to the radiolabelled primer. Transcription ends one nucleotide before the start codon (usually AUG) of the coding region. Such positions defining the region of transcription is referred to as the “transcription boundaries.”

Conditional use of sigma factors—transcription units can be transcribed in a condition-dependent manner through alternative sigma factor use. The genome-scale location map of sigma factors provides basic information to design the tunable/controllable/regulatable promoters. For example, the genome-scale location of all sigma factors in E. coli has been determined in this invention.

Selection of sigma factors, TSSs or 5′UTR sequences—from the sigma factor interation network, the house-keeping sigma factor or alternative sigma factors can be selected for obtaining the optimal or suboptimal biochemical reaction network properties. From the reporter vector library, the alternative TSSs or 5′UTR sequences can be selected for obtaining the optimal or suboptimal biochemical reaction network properties. Using the selected sigma factors, TSSs or 5′UTR sequences, the native promoters of the selected genes or transcription units in the genome can be genetically manipulated. Alternatively, instead of the manipulation of native genome, the vectors comprising alternative TSSs and 5′UTR sequences can be used to achieve the optimal or suboptimal biochemical reaction properties.

Conditional use of alternative TSSs—transcription units can be transcribed in a condition-dependent manner through alternative TSS use. The use of alternative TSS can be determined by the novel 5′-RACE-seq method using a unique RNA adapter and massive-scale sequencing. For example, 4,133 TSSs were determined in E. coli genome. 35% of promoters contain multiple TSSs, representing the presence of alternative TSSs for large portions of the E. coli transcription units

As used herein, the term “re-sequencing” or “resequencing” refers to a technique that determines the sequence of a genome of an organism using a reference sequence that has already been completely determined. It should be understood that resequencing may be performed on both the entire genome of an organism or a portion of the genome large enough to include the genetic change of the organism as a result of selection.

As used herein, the term “genetic material” refers to the DNA within an organism that is passed along from one generation to the next. Normally, genetic material refers to the genome of an organism. Extra-chromosomal, such as organelle or plasmid DNA, can also be a part of the ‘genetic material’ that determines organism properties. As used herein, “regulatory region,” when used in reference to a gene or genome, refers to a DNA sequence that controls gene expression. As used herein, a “gene product” refers to biochemical material, either RNA or protein, resulting from expression of a gene. Thus, a measurement of the amount of gene product is sometimes used to infer how active a gene is.

As used herein, the term “genetic change” or “genetic adaptation” refers to one or more mutations within the genome of an organism. As used herein, the term “mutation” refers to a difference in the sequence of DNA nucleotides of two related organisms, including substitutions, deletions, insertions and rearrangements, or motion of mobile genetic elements, for example. The term “introduction,” as used herein, refers to the putting of something such as a genetic change into something else, such as an organism. As such, the term “mutagenesis” is intended to mean the introduction of genetic change(s) into an organism.

The terms “polypeptide,” “peptide” and “protein” are used interchangeably herein to refer to two or more amino acid residues joined to each other by peptide bonds or modified peptide bonds. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer. “Polypeptide” refers to both short chains, commonly referred to as peptides, oligopeptides or oligomers, and to longer chains, generally referred to as proteins. Polypeptides may contain amino acids other than the 20 gene-encoded amino acids. Likewise, “protein” refers to at least two covalently attached amino acids, which includes proteins, polypeptides, oligopeptides and peptides. A protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes amino acid residues such as proline and hydroxyproline. The side chains may be in either the (R) or the (S) configuration. Thus, the term “proteomics,” as used herein, refers to the large-scale study of proteins, particularly their structures and functions.

As used herein, the terms “ChIP-on-chip” or “ChIP-chip” refer to a technique that combines chromatin immunoprecipitation (“ChIP”) with microarray technology (“chip”). Like regular ChIP, ChIP-on-chip is used to investigate interactions between proteins and DNA in vivo. Specifically, it allows the identification of the cistrome, sum of binding sites, for DNA-binding proteins on a genome-wide basis. Whole-genome analysis can be performed to determine the locations of binding sites for almost any protein of interest.

As used herein, the term “tiling array” refers to a subtype of a microarray wherein probes are short fragments that are designed to cover the entire genome or contiguous regions of the genome. Depending on the probe lengths and spacing, different degrees of resolution can be achieved. The number of features on a single array can range from 10,000 to greater than 6,000,000, with each feature containing millions of copies of one probe. Traditional DNA microarrays designed to look at gene expression use a few probes for each known or predicted gene. In contrast, tiling arrays can produce an unbiased look at gene expression because previously unidentified genes can still be incorporated.

As used herein, the term “deep sequencing” refers to the next-generation of sequencing technologies that generate huge numbers of sequencing reads per experiment or instrument run. These sequencing-based approaches have some distinct advantages over microarray-based approaches for genome-wide transcriptomics (the study of gene expression) and epigenomics (the study of chromatin organization and dynamics), such as avoiding complex intermediate cloning and microarray construction steps and the ability to generate a massive amount of sequence quickly. Using these approaches, gene expression is assayed by directly sequencing cDNA molecules obtained from an mRNA sample and simply counting the number of molecules corresponding to each gene to assess transcript abundance. Exemplary techniques included within the term “deep sequencing” include, but are not limited to, massively parallel signature sequencing (MPSS), sequencing by synthesis (SBS), 454 Life Sciences' SBS pyrosequencing method, Applied Biosystems' SOLiD sequencing by ligation system, and Helicos Biosciences' single-molecule synthesis platform.

As used herein, the term “external environment” refers to the environment surrounding the organism. Examples of the external environment include, but are not limited to, nature, laboratory culture media, a surface or a mammal.

As used herein, the terms “selected environment,” “condition” or “conditions” refer to any external property that causes an organism to genetically adapt, evolve, change or mutate for survival. Exemplary “conditions” or “environments” include, but are not limited to, a particular medium, volume, vessel, temperature, mixing, aeration, gravity, electromagnetic field, cell density, pH, nutrients, phosphate source, nitrogen source, symbiosis with one or more organisms, and interaction with a single species of organism or multiple species of organisms (i.e., a mixed population). Also included as “conditions” or “environments” are substances that are toxic to the organism, such as heavy metals, antibiotics and chlorinated compounds. It should be understood that time may also be considered a “condition” since organisms are not static entities. Thus, a culture grown over an extended period of time (e.g., days, weeks, months, years) may produce different strains over the course of its genetic adaptation. An exemplary period of time is 4 to 180 days.

As used herein, the term “clone” refers to a single cell or population of cells that originated from a single cell. A clone is known to consist of cells with only one genotype or to have had a single genotype previously. The term “population” is intended to mean a group of individuals or cells. A “mixed population” therefore refers a group of cells from multiple species or to the collective genomes of naturally occurring organisms.

As used herein, the term “medium” or “media” refers to the chemical environment to which an organism is subjected or is provided access. The organism may either be immersed within the media or be within physical proximity thereto. Media are typically composed of water with other additional nutrients and/or chemicals that may contribute to the growth or maintenance of an organism. The ingredients may be purified chemicals (i.e., “defined” media) or complex, uncharacterized mixtures of chemicals such as extracts made from milk or blood. Standardized media are widely used in laboratories. Examples of media for the growth of bacteria include, but are not limited to, LB and M9 minimal medium. The term “minimal” when used in reference to media refers to media that support the growth of an organism, but are composed of only the simplest possible chemical compounds. For example, M9 minimal medium is composed of the following ingredients dissolved in water and sterilized: 48 mM Na₂HPO₄, 22 mM KH₂PO₄, 9 mM NaCl, 19 mM NH₄Cl, 2 mM MgSO₄, 0.1 mM CaCl₂, 0.2% carbon and energy source (e.g., glucose).

As used herein, the term “culture” refers to medium in a container or enclosure with at least one cell or individual of a viable organism, usually a medium in which that organism can grow. As used herein, the term “continuous culture” is intended to mean a liquid culture into which new medium is added at some rate equal to the rate at which medium is removed. Conversely, a “batch culture,” as used herein, is intended to mean a culture of a fixed size or volume to which new media is not added or removed.

As used herein, the term “culture conditions” refers to the conditions of the external environment. The culture conditions may be altered to produce an effect. For example, changing the media used to grow bacteria to examine the results would result in changing the culture conditions.

The term “organism” refers both to naturally occurring organisms and to non-naturally occurring organisms, such as genetically modified organisms. An organism can be a virus, a unicellular organism, or a multicellular organism, and can be either a eukaryote or a prokaryote. Further, an organism can be an animal, plant, protist, fungus or bacteria. Exemplary organisms include, but are not limited to bacterial organisms, which include a large group of single-celled, prokaryote microorganisms, and archeal organisms, which include a group of single-celled microorganisms. Bacterial organisms also include gram negative bacteria, gram positive bacteria, pathogenic bacteria, electrosynthetic bacteria and photosynthetic bacteria. Additional examples of bacterial organisms include, but are not limited to, Acinetobacter baumannii, Acinetobacter baylyi, Bacillus subtilis, Buchnera aphidicola, Chromohalobacter salexigens, Clostridium acetobutylicum, Clostridium beijerinckii, Clostridium thermocellum, Corynebacterium glutamicum, Dehalococcoides ethenogenes, Escherichia coli, Francisella tularensis, Geobacter metallireducens, Geobacter sulfurreducens, Haemophilus influenza, Helicobacter pylori, Klebsiella pneumonia, Lactobacillus plantarum, Lactococcus lactis, Mannheimia succiniciproducens, Mycobacterium tuberculosis, Mycoplasma genitalium. Neisseria meningitides, Porphyromonas gingivalis, Pseudomonas aeruginosa, Pseudomonas putida, Rhizobium etli, Rhodoferax ferrireducens, Salmonella typhimurium, Shewanella oneidensis, Staphylococcus aureus, Streptococcus thermophiles, Streptomyces coelicolor, Synechocystis sp. PCC6803, Thermotoga maritime, Vibrio vulnificus, Yersinia pestis, Zymomonas mobilis, Halobacterium salinarum, Methanosarcina barkeri, Methanosarcina acetivorans, Methanosarcina acetivorans, Natronomonas pharaonis, Arabidopsis thaliana, Aspergillus nidulans, Aspergillus niger, Aspergillus oryzae, Cryptosporidium hominis, Chlamydomonas reinhardtii.

As used herein the term “amino acid metabolism” refers to any biological process that involves an amino acid. Examples of such processes include but are not limited to the biosynthesis of amino acids from precursor molecules, the transport of an amino acid into, out of or within an organism and utilization of amino acids in metabolic processes in the organism. Amino acids are well known in the art and include but are not limited to alanine, asparagine, aspartic acid, cysteine, glutamic acid, glutamine, glycine, histidine, isoleucine, leucine, lysine, methionine, phenylalanine, proline, serine, threonine, tryptophan, tyrosine, valine, ornithine, selenocysteine and taurine.

As used herein, the term “regulatory motif” refers to a DNA binding sequence or site to which transcription factors bind. The regulatory motif is associated with the activation, suppression, up regulation and down regulation of genes in response to transcription factor binding. In one aspect of the present invention the regulatory motif denotes a DNA binding sequence or site for transcription factors associated with amino acid metabolism.

As used herein, the term “small molecule” is a molecule that modulates amino acid metabolism in an organism. Examples of small molecules includes, but is not limited to, amino acids, nucleotides, nutrients and trace elements. In one aspect of the invention, the small molecule is an amino acid. In other aspects the small molecule is a molecule that produces modulation in the amino acid metabolism of an organism in a similar manner to an amino acid.

As used herein, the term “transcription factor” or “TF” means is a protein that binds to specific DNA sequences, thereby controlling the flow (or transcription) of genetic information from DNA to mRNA. A defining feature of transcription factors is that they contain one or more DNA-binding domains (DBDs), which attach to specific sequences of DNA adjacent to the genes that they regulate. Transcription factors include but are not limited to, be ArgR, Lrp, TrpR, TyrR, PurR, PyrR, Fnr, ArcA, Crp, Cra, DgsA, Fis, Hns, HU, Ihf, StpA and Dps. Transcription factors may also include acrR, ampD; appR; appY; araC; arcA; argR; ascG; asnC; atoC; baeR; baeS; barA; basS; bglG (bglC, bglS); birA (bioR, dhbB); btuR; cadC; celD; chaB; chaC; cpxR; crl; cspA; cspE; csrA; cynR; cysB; dadQ (alnR); dadR (alnR); deoR (nucR, tsc, nupG); dgoR; dicA; dnaK (gro, groP, groPAB, groPC, groPF, grpC, grpF, seg); dniR; dsdC; ebgR; envY; envZ (ompB, perA, tpo); evgA; evgS; exuR; fadR (dec, ole, thdB); fed; fecR; fhlA; fhlB; fimB (pil); fimE (pilH); flhC (flal); flhD (flhB); fliA (flaD, rpoF); fnr (frdB, nirA, nirR); fruR (fruC, shl); fucR; fur; gadR gene product from Lactococcus lactis; galR; galS (mglD); gatR; gcvA; glgS; glnB; glnG (gln, ntrC); glnL (glnR, ntrB); glpR; gltF; gntR; hha; himD (hip); hrpB gene product from Pseudomonas solanacearum; hybF; hycA; hydG; hydH; iciA; iclR; ileR (avr, flrA); ilvR; ilvU; ilvY; inaA; inaR; kdgR; leuO; leuR; leuY; lexA; lldR (lctR); lpp; IrhA (genR); lrp (ihb, livR, Iss, lstR, oppl, rblA, mbf); lysR; malI; malT (malA); marA (cpxB, soxQ); marR; melR; metJ; metR; mglR (R-MG); mhpR; mhpS; micF (stc); mprA (emrR); mtlR; nagC (nagR); narL (frdR, narR); narP; nhaR; ompR (cry, envZ, ompB); oxyR (mor, momR); pdhR; phnF; phoB (phoRc, phoT); phoP; phoQ; phoR (R1pho, nmpB, phoR1); phoU (phoT); poaR; poxA; proQ; pspA; pspB; pspC; pssR; purR; putA (poaA) gene product from Salmonella enterica serotype Typhimurium; pyrI; rbsR; rcsA; rcsB; rcsC; rcsF; relB; rfaH (sfrB); rhaR; rhaS; rnk; rob; rseA (mclA); rseB; rseC; rspA; rspB; rssA; rssB; sbaA; sdaC; sdiA; and serR.

As used here in the term “pathway” or “biologic pathway” or “biochemical pathway” is a series of actions among molecules in a cell that leads to a certain product or a change in a cell. Such a pathway can trigger the assembly of new molecules, turn genes on and off, or spur a cell to move. Some of the most common biological pathways are involved in metabolism, the regulation of genes and the transmission of signals. Such pathways include the binding of transcription factors to DNA to regulate the biosynthesis of amino acids, the transport of amino acids and the utilization of amino acids.

As used herein, the term “biosynthesis” or “biosynthesis pathway” means any pathway involved in the biosynthesis of amino acids. This includes proteins and genes which are modulated, activated, suppressed, up regulated or down regulated during the biosynthetic process. Such genes include, but are not limited to, aroB, aroK. aroH, aroA, tyrB, tyrA, aroF, aroL, aroG, trpABCDE, carAB, argCBH, argA, araF, argG, argD, argE, argI, dapE, hisLGDCBHAFI, gdhA, gltBD, leuABCD, ilvIH, avtA, thrLABC, serC, serA, serB and ansB. One or more of these genes may be up regulated during the biosynthesis of amino acids. For example, ArgR regulates the transcription of all of the genes involved in the biosynthesis of arginine and histidine. The genes gltBD, aroB, aroK and dapE are involved in the biosynthesis of glutamate, aromatic amino acids and lysine, respectively. The genes encoding the enzymes for the biosynthesis of branched chain amino acids were comprehensively regulated by Lrp, which also controls the transcription of gltBD and gdhA encoding glutamate synthase and glutamate dehydrogenase (glutamate biosynthesis), serC and serB encoding phosphoserine transaminase and phosphatase (serine biosynthesis), thrABC operon for aspartate kinase, homoserine kinase, and threonine synthase (threonine biosynthesis), argA for N-acetylglutamate synthase (arginine biosynthesis), and aroA for 3-phosphoshikimate-1-carboxyvinyltransferase (the chorismate formation for aromatic amino acid biosynthesis). TrpR regulates the transcription of genes involved in tryptophan biosynthetic pathway (trpLEDCBA operon), as well as aroH and aroL. In addition, it has been determined that TyrR directly regulates several genes in the aromatic amino acid biosynthesis (aroF, aroG, aroK, aroA, tyrA, and tyrB) in response to exogenous tyrosine.

As used herein, the term “transportation” or “transportation pathway” means any pathway involved in the transport of amino acids into an organism from the external environment, the transport of amino acids from an organism to the external environment or the transport of amino acids within the organism. This includes proteins and genes which are modulated, activated, suppressed, up regulated or down regulated during the transportation process. Examples of genes which are modulated during the transportation pathway include, but are not limited to, aroP. tyrP, mtr, artJ, artMQIP, gltP, brnQ, livKHMGF, livJ, cycA, tdcC, sdaC, sstT, proY, potFGHI, dtpB, dppABCDF, oppABCDF, lolCDE, mdtL, eutR and ycaM.

As used herein, the term “utilization” or “utilization pathway” means any pathway involved in the utilization of amino acids within the organism. Amino acids are primarily used in the synthesis of proteins. However, amino acids are also utilized roles as metabolic intermediates, such as in the biosynthesis of the neurotransmitter gamma-aminobutyric acid. Many amino acids are used to synthesize other molecules, for example: Tryptophan is a precursor of the neurotransmitter serotonin, Tyrosine (and its precursor phenylalanine) are precursors of the catecholamine neurotransmitters dopamine, epinephrine and norepinephrine Glycine is a precursor of porphyrins such as heme, Arginine is a precursor of nitric oxide. Ornithine and S-adenosylmethionine are precursors of polyamines, Aspartate, glycine, and glutamine are precursors of nucleotides, Phenylalanine is a precursor of various phenylpropanoids, which are important in plant metabolism. This includes proteins and genes which are modulated, activated, suppressed, up regulated or down regulated during the transportation process. Examples of genes which are modulated during the utilization pathway include, but are not limited to, fadJ, tnaA, astCADBE, aspA, ilvE, tdcB, tdh-kbl, sdaA, sdaB, ivlA, dadAX, metK, puuEB, pepD and eutBCLK.

In one embodiment, the present invention is a method of identifying a regulatory motif for amino acid metabolism in a target organism comprising (a) obtaining the full genome sequence a target organism; (b) obtaining the genome-wide binding of a transcription factor from the organism; (c) obtaining the sequence of the binding sites from the organism; (d) obtaining the data described in (b) and (c) under a series of culture conditions for the organism; and (e) iteratively mapping the data sets described in (d) onto the DNA sequence in (a) and identify binding sites associated with genes involved in amino acid metabolism, thereby identifying the regulatory motif for amino acid metabolism in the target organism.

A test organism can be a virus, a unicellular organism, or a multicellular organism, and can be either a eukaryote or a prokaryote. Further, a test organism can be an animal, plant, protist, fungus or bacteria. Exemplary test organisms include, but are not limited to bacterial organisms, which include a large group of single-celled, prokaryote microorganisms, and archeal organisms, which include a group of single-celled microorganisms. In one aspect, the target organism is a bacterial organism. Bacterial organisms also include gram negative bacteria, gram positive bacteria, pathogenic bacteria, electrosynthetic bacteria and photosynthetic bacteria. In a further aspect, the target organism may be Acinetobacter baumannii, Acinetobacter baylyi, Bacillus subtilis, Buchnera aphidicola, Chromohalobacter salexigens, Clostridium acetobutylicum, Clostridium beijerinckii, Clostridium thermocellum, Corynebacterium glutamicum, Dehalococcoides ethenogenes, Escherichia coli, Francisella tularensis, Geobacter metallireducens, Geobacter sulfurreducens, Haemophilus influenza, Helicobacter pylori, Klebsiella pneumonia, Lactobacillus plantarum, Lactococcus lactis, Mannheimia succiniciproducens, Mycobacterium tuberculosis, Mycoplasma genitalium. Neisseria meningitides, Porphyromonas gingivalis, Pseudomonas aeruginosa, Pseudomonas putida, Rhizobium etli, Rhodoferax ferrireducens, Salmonella typhimurium, Shewanella oneidensis, Staphylococcus aureus, Streptococcus thermophiles, Streptomyces coelicolor, Synechocystis sp. PCC6803, Thermotoga maritime, Vibrio vulnificus, Yersinia pestis, Zymomonas mobilis, Halobacterium salinarum, Methanosarcina barkeri, Methanosarcina acetivorans, Methanosarcina acetivorans, Natronomonas pharaonis, Arabidopsis thaliana, Aspergillus nidulans, Aspergillus niger, Aspergillus oryzae, Cryptosporidium hominis, Chlamydomonas reinhardtii. In an additional aspect the target organism is E. coli.

In an additional aspect, the genome-wide binding of the transcription factor is obtained by chromatin immunoprecipitation coupled with a microarray. In a further aspect, the genome-wide binding of the transcription factor is obtained by deep sequencing of immunoprecipitated DNA. In an aspect, the sequence of the binding sites is obtained using tiled expression arrays. In a further aspect, the sequence of the binding sites is obtained using deep sequencing of the isolated DNA. In an additional aspect, the regulatory motif is associated with amino acid transport, biosynthesis or utilization. Further, the transcription factor may be selected from the group consisting of: ArgR, Lrp and TrpR. In one aspect, one or more small molecules is used to produce the series of culture conditions. Further, the small molecule may be an amino acid.

In an additional embodiment, the present invention is a regulatory motif for ArpR binding selected from the group consisting of SEQ ID NOs:1-126. In another embodiment, the present invention is a regulatory motif for Lrp-L binding selected from the group consisting of SEQ ID NOs:127-265. In a further embodiment, the present invention is a regulatory motif for TrpR binding selected from the group consisting of SEQ ID NOs:266-279.

One embodiment of the of the present invention is a method of modulating ArgR activity comprising contacting ArgR with a small molecule. In one aspect, the small molecule is an amino acid. Further, the amino acid may be selected from the group consisting of phenylalanine, tyrosine, tryptophan, lysine, arginine, histidine, aspartic acid, glutamic acid, valine, isoleucine, leucine, alanine, glycine, serine, threonine and proline. In an aspect, the modulated activity is activation or repression of at least one pathway. Further, the pathway maybe an amino acid transportation, biosynthesis or utilization pathway. In one aspect, the amino acid is phenylalanine, tyrosine or tryptophan and the modulated activity is activation of transportation or utilization pathway and repression of a biosynthesis pathway. In an additional aspect, the amino acid is lysine, arginine or histidine and the modulated activity is activation of a utilization pathway and repression of a biosynthesis and a transportation pathway. In a further aspect, the amino acid is asparagine or glutamine and the modulated activity is activation of transportation pathway and repression of a utilization and a biosynthesis pathway.

In one embodiment, the present invention is a method of modulating Lrp activity comprising contacting Lrp with a small molecule. In one aspect, the small molecule is an amino acid. Further, the amino acid may be selected from the group consisting of phenylalanine, tyrosine, tryptophan, lysine, arginine, histidine, aspartic acid, glutamic acid, valine, isoleucine, leucine, alanine, glycine, serine, threonine and proline. In an aspect, the modulated activity is activation or repression of a at least one pathway. Further, the pathway maybe an amino acid transportation, biosynthesis or utilization pathway. In an aspect, the modulated activity is activation or repression of at least one pathway. Further, the pathway maybe an amino acid transportation, biosynthesis or utilization pathway. In one aspect, the amino acid is phenylalanine, tyrosine or tryptophan and the modulated activity is activation of transportation or utilization pathway and repression of a biosynthesis pathway. In another aspect, the amino acid is lysine, arginine or histidine and the modulated activity is activation of a transportation or utilization pathway and repression of a biosynthesis pathway. In a further aspect, the amino acid is asparagine or glutamic acid and the modulated activity is activation of a utilization or a biosynthesis pathway and repression of a transportation pathway. In yet another aspect, the amino acid is valine, isoleucine or leucine and the modulated activity is activation of a transportation or a biosynthesis pathway and repression of a utilization pathway. In an aspect, the use wherein the amino acid is alanine, glycine, serine, threonine or proline and the modulated activity is activation of a transportation or a utilization or a pathway and repression of a biosynthesis pathway.

In an embodiment, the present invention is an amino acid regulatory motif comprising the activation of a transportation and biosynthesis pathway and repression of a utilization pathway. In an additional aspect, the present invention is an amino acid regulatory motif comprising the activation of a biosynthesis pathway and repression of a biosynthesis and a utilization pathway. In a further embodiment, the present invention is an amino acid regulatory motif comprising the activation of a biosynthesis and utilization pathway and repression of a transportation pathway.

In one embodiment the present invention is the use of a small molecule to modulate amino acid metabolism. The methods described herein can be used to screen for small molecules which modulate amino acid metabolism. Such molecules may be useful in inhibiting the growth of the organism.

As is known in the art, bioinformatic or computational methods are used to find elements on a genomic sequence. However, the algorithms used today are based on information that has been experimentally determined in a reference organism(s). The output from the execution of such algorithms is thus a prediction based on extrapolation of information from one or more reference genomes. Since such predictions may or may not be accurate, the determination of the regulatory motifs for amino acid metabolism, as described herein, leads to correction of such potentially inaccurate sequence-based annotations because the information is directly measured and determined for the genome for which the regulatory motifs for amino acid metabolism is built.

The following examples are intended to illustrate but not limit the invention.

### Example 1

### Regulatory Motif Determination

This example demonstrates the detailed procedures used by describing how a specific situation is processed.

Bacterial strains and growth conditions. All strains used are E. coli K-12 MG1655 and its derivatives. The E. coli strains harboring ArgR-8myc, LRP-L-8myc, and TrpR-8myc were generated as described previously (Cho, B. K. et al. (2006) Biotechniques 40, 67-72). Glycerol stock of ArgR-8myc strains were inoculated into W2 minimal medium containing 2 g/L glucose and 2 g/L glutamine, and cultured overnight at 37° C. with constant agitation. The cultures were inoculated into 50 mL of the fresh W2 minimal media in either the presence or absence of 1 g/L arginine and continued to culture at 37° C. with constant agitation to an appropriate cell density. E. coli strains harboring LRP-L-8myc and TrpR-8myc were grown in glucose (2 g/L) minimal M9 medium supplemented with or without 20 mg/L tryptophan or 10 mM leucine, respectively.

ChIP-chip—Chromatin immunoprecipitation and microarray analysis (ChIP-chip). To identify ArgR-, Lrp-, and TrpR-binding regions in vivo, the DNA bound to ArgR protein from formaldehyde cross-linked E. coli cells harboring ArgR-8myc was isolated by chromatin immunoprecipitation with the specific antibodies that specifically recognizes myc tag (9E10, Santa Cruz Biotech) (Cho, B. K. et al. (2008) Genome Res. 18, 900-910). Cells were harvested from the exponential growth conditions in the presence or absence of exogenous arginine or tryptophan. The immunoprecipitated DNA (IP-DNA) and mock immunoprecipitated DNA (mock IP-DNA) were hybridized onto the high-resolution whole-genome tiling microarrays, which contained a total of 371,034 oligonucleotides with 50-bp tiles overlapping every 25-bp on both forward and reverse strands (Cho, B. K. et al. (2008) Proc. Natl. Acad. Sci. USA 105, 19462-19467; Cho, B. K. et al. (2009) Nat. Biotechnol. 27, 1043-1049). A ChIP-chip protocol previously described was used (Cho, B. K. et al. (2008) Genome Res. 18, 900-910; Cho, B. K. et al. (2008) Methods Mol. Biol. 439, 131-145) and microarray hybridization, wash, and scan were performed in accordance with manufacturer's instruction (Roche NimbleGen).

qPCR. To monitor the enrichment of promoter regions, 1 μL immunoprecipitated DNA was used to carry out gene-specific qPCR3. The quantitative real-time PCR of each sample was performed in triplicate using iCycler™ (Bio-Rad Laboratories) and SYBR green mix (Qiagen). The real-time qPCR conditions were as follows: 25 μL SYBR mix (Qiagen), 1 μL of each primer (10 pM), 1 μL of immunoprecipitated or mock-immunoprecipitated DNA and 22 μL of ddH₂O. All real-time qPCR reactions were done in triplicates. The samples were cycled to 94° C. for 15 s, 52° C. for 30 s and 72° C. for 30 s (total 40 cycles) on a LightCycler (Bio-Rad). The threshold cycle values were calculated automatically by the iCycler™ iQ optical system software (Bio-Rad Laboratories). Primer sequences used in this study are available on request.

ChIP-chip and expression data analysis. To identify TF-binding regions, the peak finding algorithm built into the NimbleScan™ software (Roche NimbleGen) was used. Processing of ChIP-chip data was performed in three steps: normalization, IP/mock-IP ratio computation (log base 2), and enriched region identification. The log₂ ratios of each spot in the microarray were calculated from the raw signals obtained from both Cy5 and Cy3 channels, and then the values were scaled by Tukey bi-weight mean34. The log₂ ratio of Cy5 (IP DNA) to Cy3 (mock-IP DNA) for each point was calculated from the scanned signals. Then, the bi-weight mean of this log₂ ratio was subtracted from each point. Each log ratio dataset from duplicate samples was used to identify TF-binding regions using the software (width of sliding window=300 bp). The approach to identify the TF-binding regions was to first determine binding locations from each data set and then combine the binding locations from at least five of six datasets to define a binding region using the recently developed MetaScope software. Raw gene expression CEL files were gathered from GEO for ArgR with accession GSE4724 and for LRP-L from a previous study (Cho, B. K. et al. (2008) Proc. Natl. Acad. Sci. USA 105, 19462-19467). They were normalized using background corrected robust multi-array average (Wu, Z., et al. (2004) J Am. Stat. Assoc., 99, 909-917) implemented in the R affy package. To detect differential expression between the wild type and TF deletion strains a two-tailed unpaired students t-test was applied with Microsoft excel between the experimental triplicates for the wild type and gene deletion strains. This was followed by a false discovery rate (FDR) (Benjamini, Y. et al. (1995) J. Roy. Stat. Soc. B 57, 289-300) adjustment using the R statistical software package. Before performing the FDR correction all genes which exhibited an expression level below the background across all experiments were removed. The background level was calculated as the average expression level across all intergenic probes. Only genes meeting a 5% FDR-adjusted P-value cut-off were considered to be differentially expressed. To make calls for activation or repression the methodology laid out previously was used (Cho, B. K. et al. (2008) Proc. Natl. Acad. Sci. USA 105, 19462-19467).

Motif searching. The ArgR-, LRP-L-, and TrpR-binding motif analysis was completed using the MEME and FIMO tools from the MEME software suite (Bailey, T. L. et al. (2009) Nucleic Acids Res. 37, 202-208). The proper binding motif was first determined and then scanned the full genome for its presence. The elicitation of the motif was done using the MEME program on the set of sequences defined by the ArgR-, Lrp-, and TrpR-binding regions respectively (Bailey, T. L. et al. (2006) Nucleic Acids Res. 34, 369-373). Using default settings the previously determined ArgR (Makarova, K. S. et al. (2011) Genome Biol. 2, 235-242), Lrp (Cho, B. K. et al. (2008) Proc. Natl. Acad. Sci. USA 105, 19462-19467), and TrpR (Yang, J. et al. (1996) J. Mol. Biol. 258, 37-52) motif were recovered and then tailored to the correct size by setting the width parameter to 18-bp, 15-bp, and 8-bp respectively. These motifs were then used and the PSPM (position specific probability matrix) generated for each by MEME to rescan the entire genome with the FIMO program. The sequence logo was generated from these sites.

### Example 2

### Regulatory Motif Determination of E. coli K-12 MG1655

This example demonstrates data integration and analysis to determine the regulatory motif of the E. coli K-12 MG1655 genome.

Genome-wide TF-binding regions: Regulatory code analysis

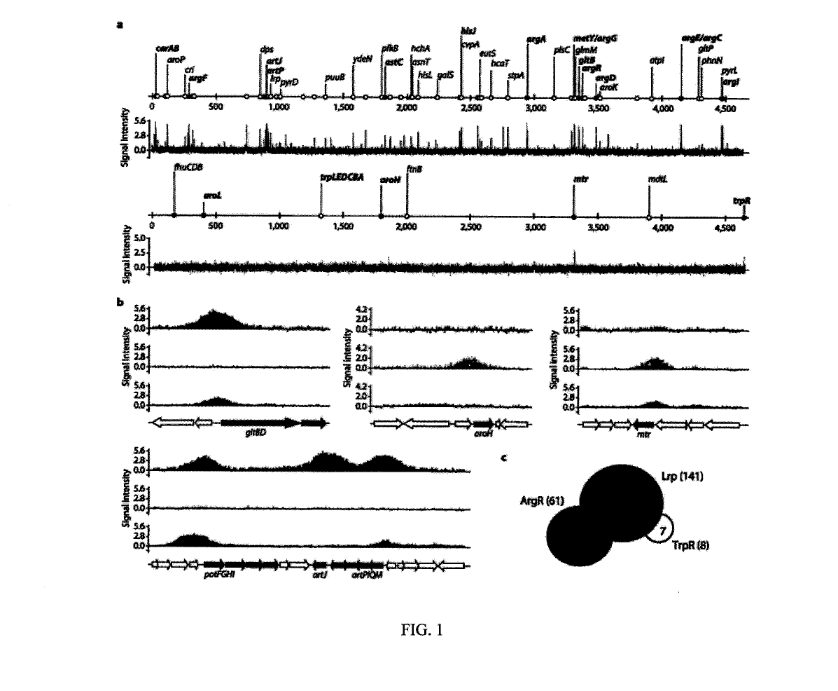

ArgR, Lrp, and TrpR are TFs involved in amino acid metabolism in E. coli, responding to arginine, leucine, and tryptophan, respectively. The binding of the small effector molecule (here being the amino acids) to these TFs carries out the genome's regulatory code by enhancing or decreasing the TFs affinity for a specific genomic region and concurrently modulating the transcription of downstream genes. In the case of LRP-L, the direct analysis of in vivo binding was fully described using chromatin immunoprecipitation coupled with microarrays (ChIP-chip) experiments. A total of 141 binding regions were analyzed, representing coverage of 74% of the previously identified regions. However, similar genome-scale data for the other two major TFs in amino acid metabolism, ArgR and TrpR were unavailable. To determine their binding regions on a genome-wide level in an unbiased manner, the ChIP-chip approach was employed to E. coli cells harboring 8.myc-tagged ArgR or TrpR protein. The resulting log₂ ratios obtained from the ChIP-chip experiments identified the genomic regions enriched in the IP-DNA sample compared with the mock IP-DNA sample and thereby represented a genome-wide map of in vivo ArgR- and TrpR-binding regions (FIG. 1a).

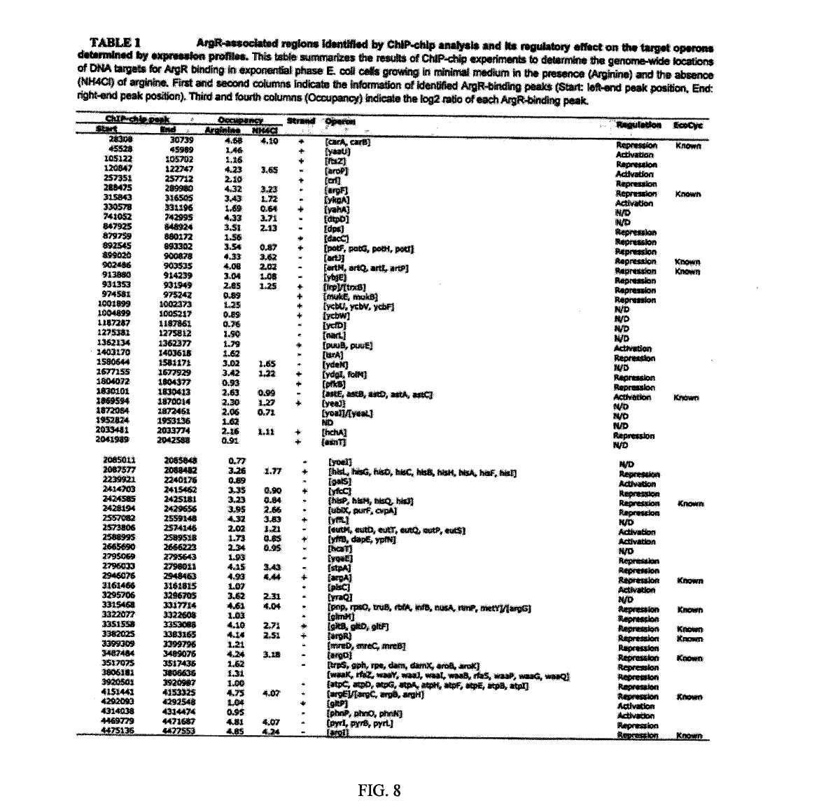

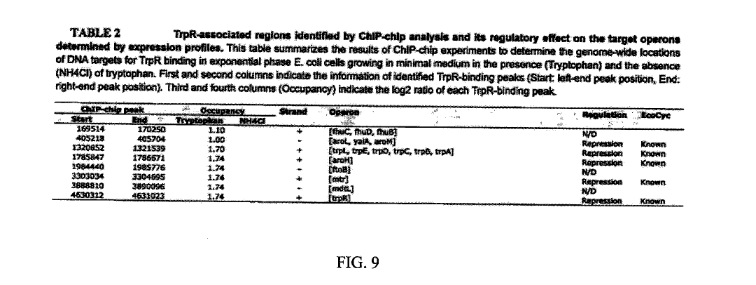

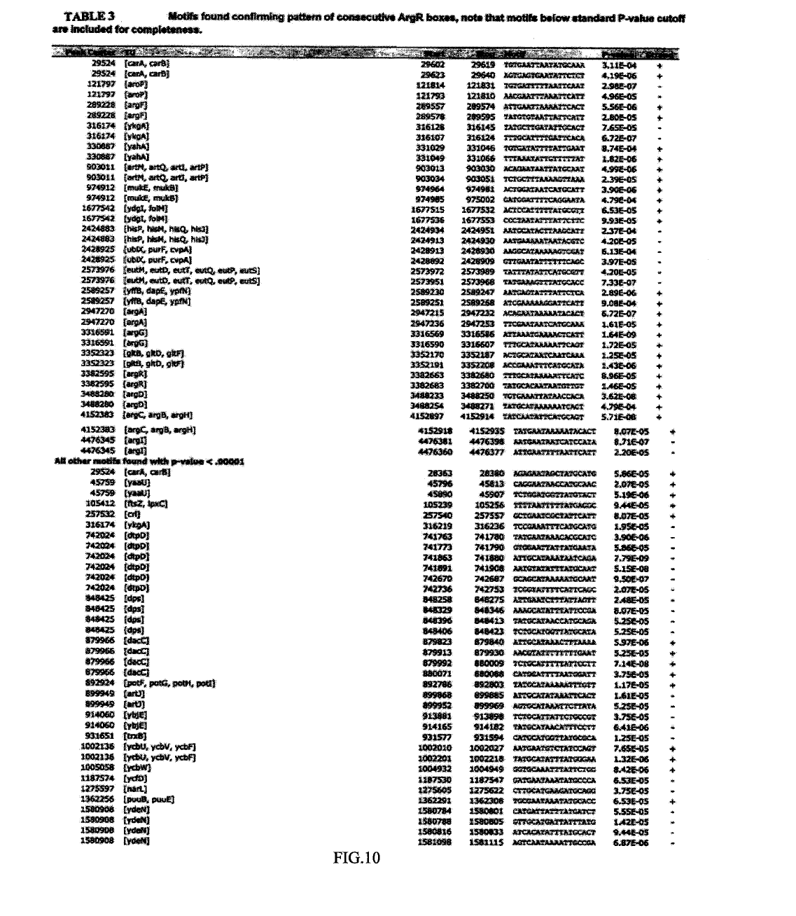

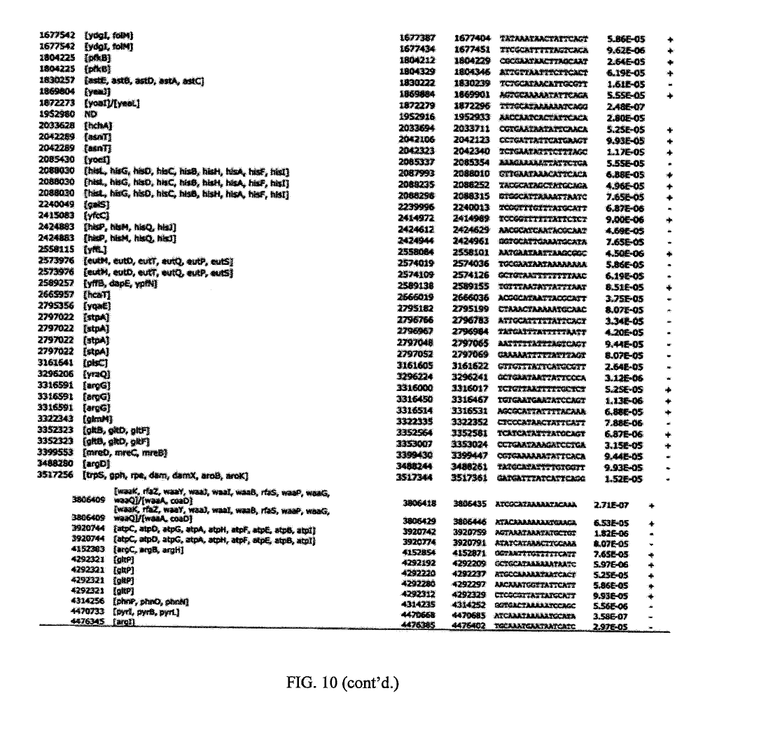

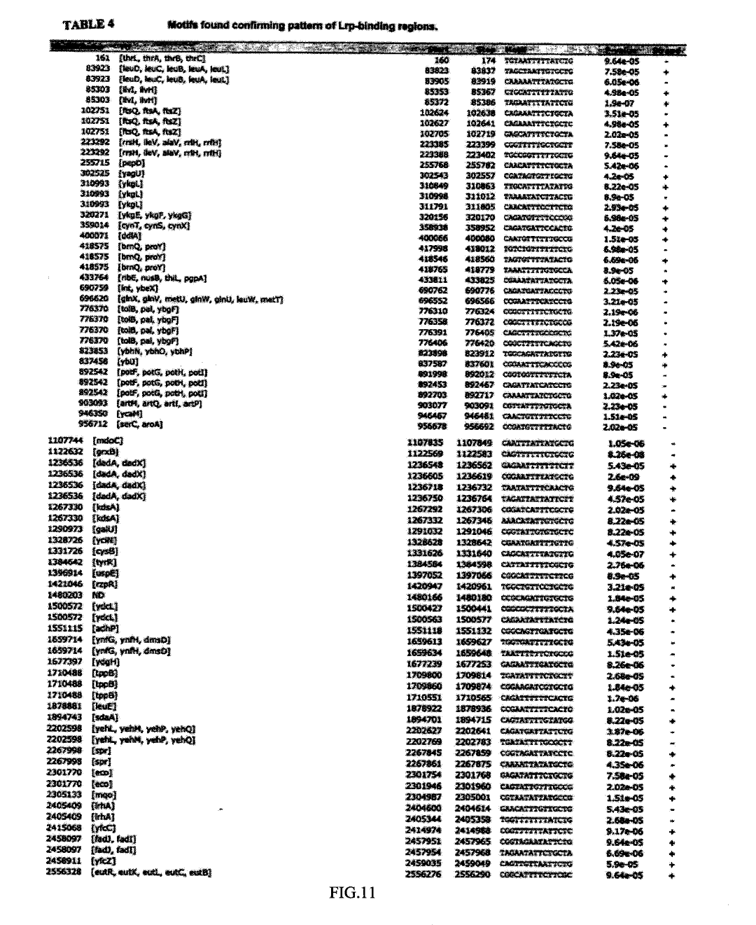

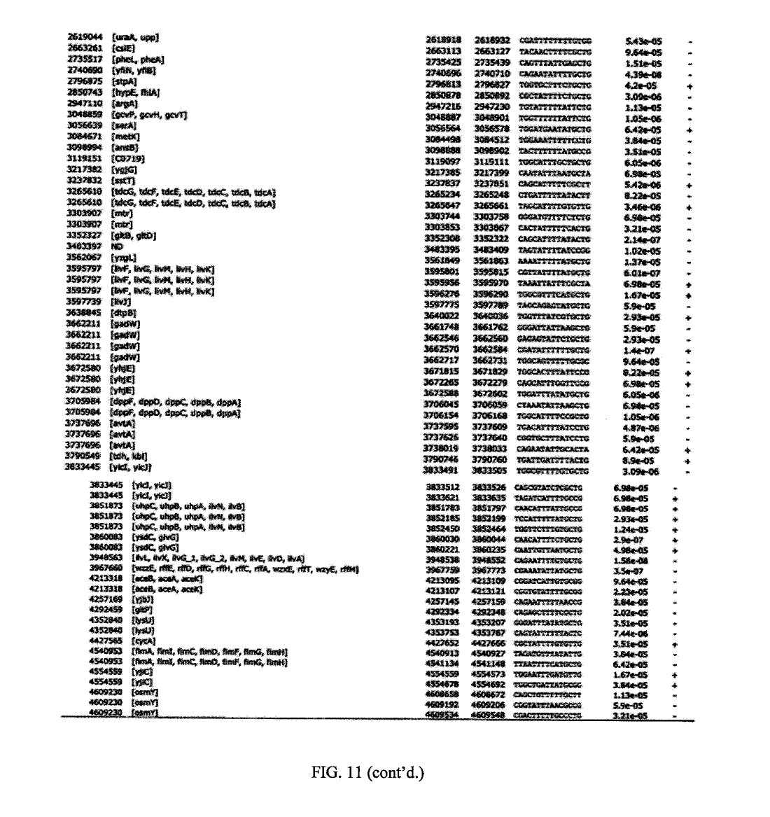

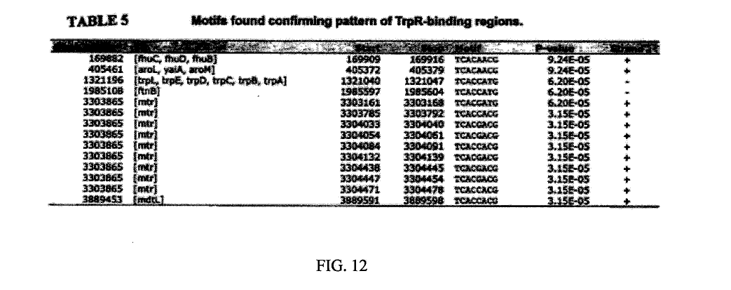

Using a previously described binding region detection algorithm (Cho, B. K. et al. (2009) Nat. Biotechnol. 27, 1043-1049), 61 and 8 unique and reproducible ArgR- and TrpR-binding regions were identified, respectively (FIGS. 8 and 9). The 61 ArgR-binding sites detected included 13 sites previously characterized by DNA-binding experiments in vitro and mutational analyses in vivo. For example, the ArgR-arginine complex transcriptionally represses gltBD, artPIQM operon, and artJ gene encoding arginine transport systems. The results confirmed that the ArgR-arginine complex binds to each of these promoter regions (FIG. 1b). In addition, the ArgR occupancy level at the promoter of the artJ gene was greater than that of artPIQM operon in the presence and absence of exogenous arginine (FIG. 8). This result is in good agreement with the de-repression/repression ratio of 28 for P_{artJ} and 3.2 for P_{artP} previously reported for repressibility of the artJ and artP promoters. Also, this result is consistent with recent microarray and qPCR experiments showing a significant arginine and ArgR-dependent down-regulation of both the artJ (about 50-fold) and artPIQM mRNA levels (about three to six-fold). In the case of TrpR, a total of five associations have been determined by DNA-binding experiments in vitro and mutational analyses in viv, all of which were also identified in the study (FIGS. 1a and 9). For instance, TrpR directly bound to the promoter regions of aroH and mtr involved in biosynthesis and transport of aromatic amino acids (FIG. 1b). Against the current genome annotation, all of the ArgR- and TrpR-binding regions were observed within intergenic regions, i.e., promoter and promoter-like regions. The same preference was observed for LRP-L-binding sites (FIGS. 8 and 9). DNA sequence motifs for each of the transcription factors were also re-derived based solely upon the ChIP binding regions and were in full agreement with previously described motifs (FIG. 7). Based on the fact that the increase in the intracellular arginine and tryptophan levels enhances ArgR and TrpR binding to its DNA targets, the confirmation of previously discovered sequence motifs, and the full coverage of the known binding regions in the data it was concluded that ArgR- and TrpR-binding regions identified here are bona fide binding sites.

Interestingly, as with gltBD, artPIQM, potFGHI, and mtr (FIG. 1b), it was observed that LRP-L directly binds to nine ArgR- and one TrpR-binding regions (FIG. 1c and FIG. 1). For example, the direct binding of Lrp to the promoter region of the gltBD operon encoding glutamate synthase resulted in the activation of its transcription. In contrast, the role of ArgR-binding represents the negative regulation of the operon. Integrating binding regions and changes in transcript levels, the reciprocal mode in the transcriptional regulation of ArgR and LRP-L was observed for cellular functions including putrescine transport (potFGHI), arginine transport (artPIQM), leucine response protein (Lrp-L), arginine biosynthesis and utilization (argA and astCADBE), the formation of nucleoid (stpA), as well as glutamate biosynthesis and transport (gltBD and gltP). While LRP-L activates the tryptophan transport (mtr), TrpR represses its transcription. In addition to confirming previously identified ArgR- and TrpR-binding regions, 48 and 3 novel ArgR- and TrpR-binding regions were found, which include the promoter region of potFGHI, encoding putrescine ABC transporter (FIG. 1b).

Identification of regulons: Topological analysis. A regulon is defined as a group of genes whose transcription is controlled by a common regulator. The arginine regulon describing the genetic and regulatory organization of the genes involved in arginine biosynthesis in E. coli was used as an example in proposing the definition of the regulon in 1964. However, it has not been included in the definition of regulon whether each regulation is direct or indirect. So far, a total of 37, 56, and 10 genes have been characterized as members of regulons directly regulated by ArgR, Lrp, and TrpR, respectively. Based upon regulatory codes described above, size of these regulons was significantly expanded and obtained 140, 283, and 15 target genes for each regulon. Since ArgR directly controls the transcription of LRP-L, the regulon size of each transcription factor can be described as ArgR (423)>LRP-L (283)>TrpR (15). These regulons represent a hierarchical structure that can be used to identify the indirect effect of the TFs. For example, thrLABC operon involved in the threonine biosynthesis was directly activated by LRP-L, either in the absence or presence of exogenous leucine. It was observed that ArgR indirectly represses this operon in response to exogenous arginine; i.e., transcriptional repression without the direct binding of ArgR. It is therefore possible to partially elucidate the indirect regulation by ArgR based on the hierarchical regulatory network. ArgR repressed LRP-L leading to the indirect repression of the thrLABC operon. As shown in this example, integrated analysis of ChIP-chip and expression profiles allowed us to fully understand the hierarchical TRN including the indirect regulatory effects.

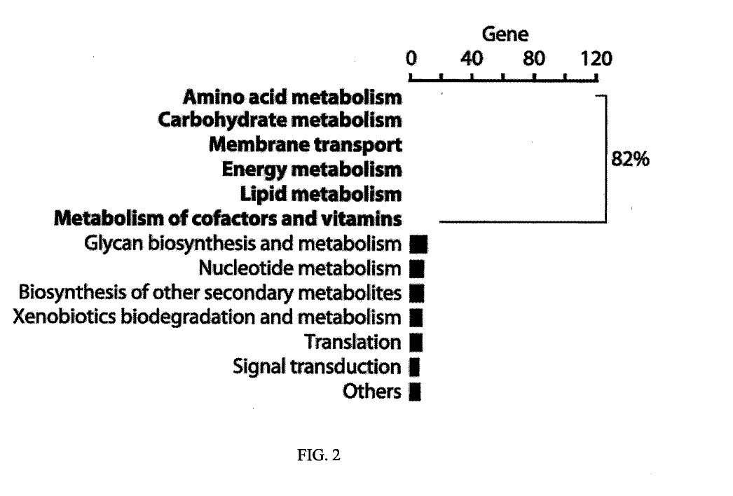

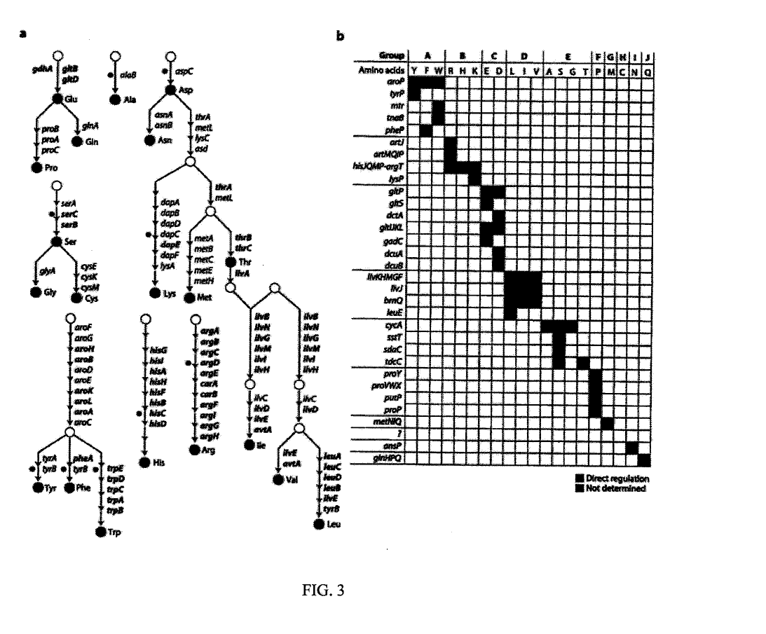

Next, 438 target genes were classified based on their functional annotation and found that most of these functions (˜82%) were assigned to amino acid metabolism and transport, as well as carbohydrate, nucleotide, and energy metabolism (FIG. 2). It was then shown (FIG. 3) that 19/20 amino acid biosynthetic pathways are directly or indirectly controlled by these three TF's. To do this directly regulated genes directly known to be involved in known amino acid biosynthetic pathways and transport systems were mapped to determine their direct metabolic roles (FIG. 3a, b). ArgR directly regulated the transcription of all genes involved in the biosynthesis of arginine and histidine. It also regulated gltBD, aroB, aroK, and dapE involved in glutamate, aromatic amino acids, and lysine biosynthesis, respectively. The genes encoding the enzymes for the biosynthesis of branched chain amino acids were comprehensively regulated by Lrp, which also controls the transcription of gltBD and gdhA encoding glutamate synthase and glutamate dehydrogenase (glutamate biosynthesis), serC and serB encoding phosphoserine transaminase and phosphatase (serine biosynthesis), thrABC operon for aspartate kinase, homoserine kinase, and threonine synthase (threonine biosynthesis), argA for N-acetylglutamate synthase (arginine biosynthesis), and aroA for 3-phosphoshikimate-1-carboxyvinyltransferase (the chorismate formation for aromatic amino acid biosynthesis). TrpR regulates the transcription of genes involved in tryptophan biosynthetic pathway (trpLEDCBA operon), as well as aroH and aroL. In addition, it has been determined that TyrR directly regulates several genes in the aromatic amino acid biosynthesis (aroF, aroG, aroK, aroA, tyrA, and tyrB) in response to exogenous tyrosine. Taken together, these four TFs controlled the biosynthesis of 12 amino acids. Furthermore, the biosynthesis of proline, glutamine, glycine, cysteine, and methionine is through branched biosynthetic pathways of glutamate, serine and aspartate (FIG. 3a). The remaining three amino acids (i.e., alanine, aspartate, and asparagine) are synthesized from glutamate as an amino donor (green dots in FIG. 3a). Therefore, biosynthetic pathways for all amino acids are directly or indirectly controlled by these four TFs.

Next, the amino acids were classified into ten groups based on the substrate specificity of each transport system, which are A (tyrosine, phenylalanine, tryptophan), B (arginine, histidine, lysine), C (glutamate, aspartate), D (leucine, isoleucine, valine), E (alanine, serine, glycine, threonine), F (proline), G (methionine), H (cysteine), I (asparagine), and J (glutamine) (FIG. 3b). This classification was based on the primary literature and EcoCyc¹⁶. As expected, the amino acids in the same group had a similar chemical structure, e.g. aromatic amino acids and branched chain amino acids in group A and group D, respectively. Transport systems for groups G-J were highly specific and therefore classified into individual groups.

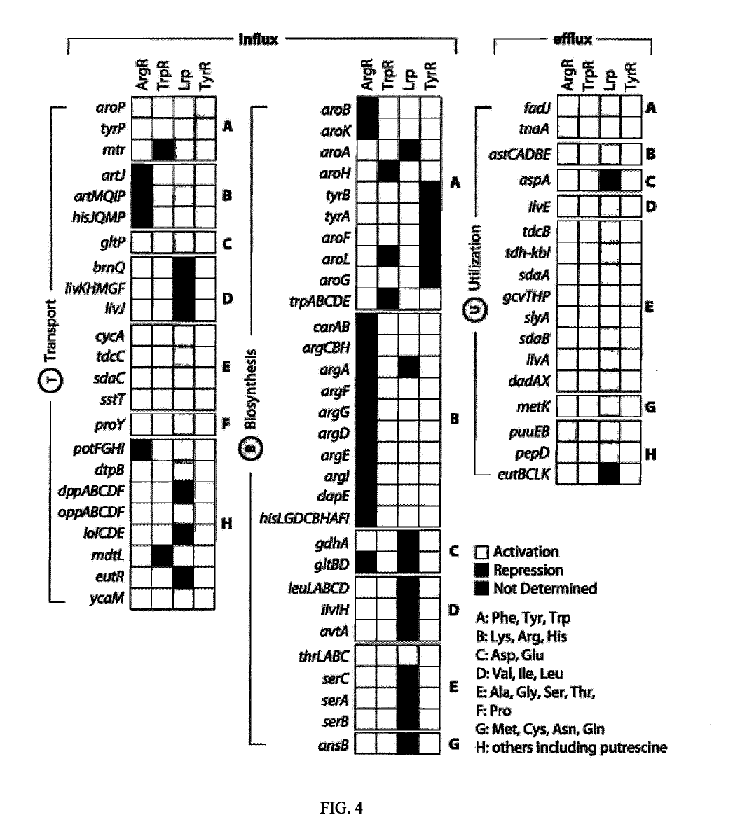

Causal relationships: Functional analysis. In general, genes for amino acid biosynthesis are repressed by each corresponding TF, whereas catabolic operons such as astCADBE, tdh-kbl, and gcvTHP are induced in response to the exogenous amino acids. To determine the causal relationships between binding of a TF and the changes in RNA transcript levels of genes in the regulons, the binding regions of ArgR, TrpR, Lrp, and TyrR were integrated with the publicly available transcriptomic data (FIG. 4) (Cho, B. K. et al. (2008) Proc. Natl. Acad. Sci. USA 105, 19462-19467; Caldara, M. et al. (2006) Microbiology 152, 3343-3354). The activation or repression was determined based upon the regulatory modes described previously. Among genes in the ArgR regulon, about 18% genes were directly activated in response to the exogenous arginine, which included aroP and gltP genes encoding aromatic amino acids and glutamate/aspartate transporters. On the other hand, ArgR repressed about 70% of its regulon members, including potFGHI, artJ, artPIQM, and hisJQMP encoding putrescine, arginine, lysine, ornithine, and histidine ABC transporters (FIG. 4). ArgR repressed genes involved in the arginine and glutamate biosynthesis pathways, and unexpectedly, it directly down-regulated genes involved in histidine, aromatic amino acids, and lysine biosynthesis pathways. In case of amino acid utilization, ArgR induced astCADBE and puuEB operons encoding the metabolic pathways for arginine and putrescine, respectively. The remaining 12% of its regulon members had a direct association with ArgR without differential gene expression. Most of the remaining genes were currently annotated as genes of unknown function (FIG. 8).

Gene expression profiles validated that Lrp directly regulates 283 genes. 45% and 55% of the LRP-L-regulated genes were repressed and activated in response to the addition of the exogenous leucine. As expected, Lrp controls the transport, biosynthetic and utilization pathways more globally than other transcription factors do. This expectation is based on the known role of Lrp as a global regulator of metabolism and nucleoid structure. Lrp represses the transport systems for branched chain amino acids (brnQ, livKHMGF, and livJ), dipeptides (dppABCDF), and lipoproteins (lolCDE) but it activates a whole set of other transporters. Transporters that are activated by Lrp are aromatic amino acids (tyrP and mtr), arginine (artMQIP), glutamate (gltP), alanine, serine, glycine and threonine (cycA, tdcC, sdaC, and sstT), proline (proY), putrescine (potFGHI), dipeptide (dtpB), and oligopeptides (oppABCDF) (FIG. 4). In terms of amino acid biosynthetic pathways, Lrp represses all genes but the thrLABC operon for threonine biosynthesis. For amino acid utilization, Lrp activates all pathways for aromatic amino acids, arginine, aspartate, branched chain aromatic amino acids, alanine, glycine, serine, threonine, methionine, and putrescine. In case of the TrpR regulon, a total of 15 genes are directly regulated, of which 13 genes are repressed (FIG. 9). TrpR also represses mtr encoding the tryptophan transporter as well as aroH, aroL, and trpABCDE involved in the tryptophan biosynthesis pathway. While TyrR activates the transport systems for aromatic amino acids (aroP, tyrP, and mtr), it represses tyrosine biosynthetic pathway comprising of aroG, aroL, aroF, tyrA, and tyrB (FIG. 4).

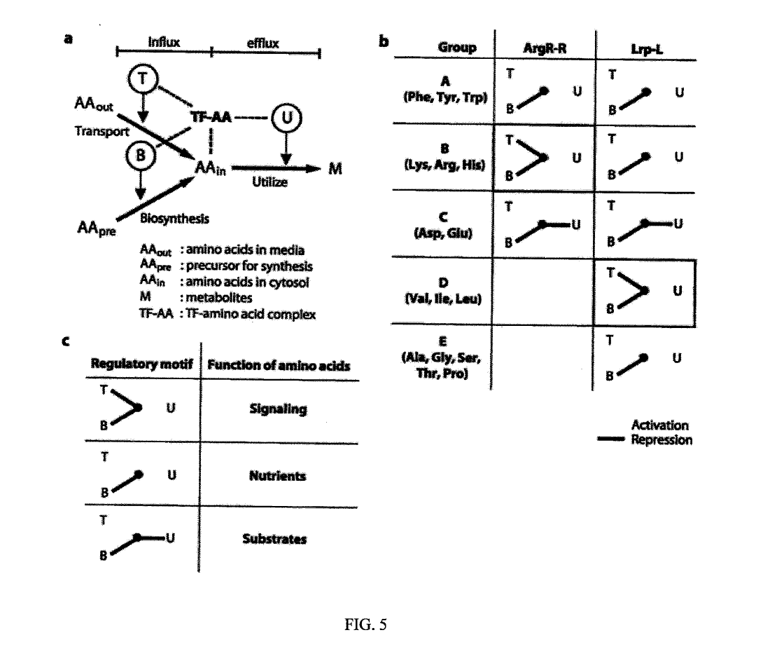

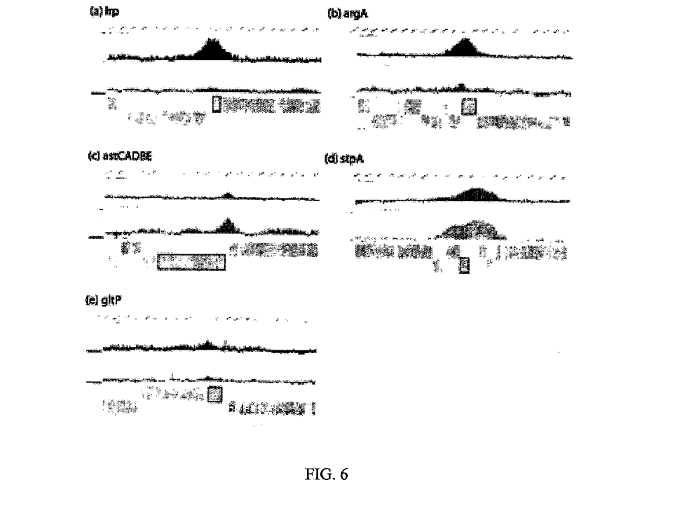

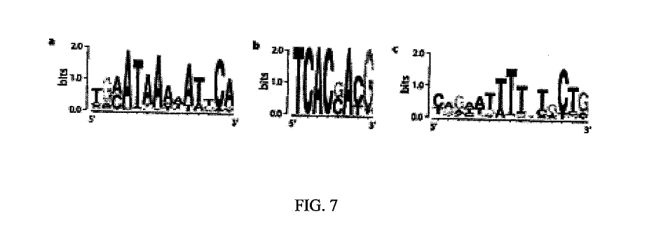

Function of a stimulon: Elucidation of regulatory logic. Based on the integrated analysis of TF-binding locations and gene expression profiles, transport, biosynthesis, and utilization of amino acids were connected, and generate the connected bidirectional circuits (FIG. 5a). In the left feed-back circuit, TF-amino acid (TF-AA) complexes regulate the transcription of the transporters (T) and biosynthesis pathways (B), facilitating the influx of the amino acid molecules (AAᵢₙ) from amino acids in the media (AAₒᵤₜ) and precursors (AAₚᵣₑ). In the right feed-forward circuit, TF-AA complexes control transcription of utilization genes (U) responsible for converting AAᵢₙ into metabolites (M). Thus, the logical structures of the connected bidirectional circuit motifs can be described by a notation that uses three signs indicating repression (R) or activation (A) for each of T, B, and U (FIG. 5b). For example, the A-R-A circuit motif indicates that the transcription of transport, biosynthesis, and metabolic genes are activated, repressed, and activated, respectively, whereas the R-R-A circuit motif demonstrates that the transcription of both transport and biosynthesis are repressed and the metabolic genes are activated. The possible logical structures of the connected circuit motifs can be characterized depending on how the TF-AA complex activates or represses both influx (T and B) and efflux (U) in response to the exogenous amino acids. Based on the connected circuit motifs, the behavior of logical structures of the transcription of transport, biosynthesis, and metabolic genes in responses to the exogenous arginine and leucine (FIG. 5b) were analyzed.

Surprisingly, there are only three influx-efflux combinations found between amino acid groups and TFs (FIG. 5c). For example, the connected circuit motif controlled by ArgR-arginine complex shows the R-R-A logical structure for group B amino acids (lysine, histidine, and arginine), whereas the logical structure of the motif is switched to A-R-R for glutamate and aspartate and A-R-A for other amino acids. On the other hand, the connected motif controlled by Lrp-Leucine complex indicates the R-R-A logical structure for group D (valine, leucine, and isoleucine) and is again switched to A-R-R for glutamate and aspartate and A-R-A for other amino acids. For glutamate the primary observation was that the utilization was repressed given its role as a substrate for nine biosynthetic pathways (FIG. 3, 4). However, that the regulation is highly complex and not universally repressed. This logically follows from the critical and centralized role it plays throughout the metabolome. Overall, it was concluded that for two global transcription factors (ArgR and Lrp) in amino acid regulation, the connected circuit motif has an R-R-A logical structure for signaling molecules (i.e., arginine for ArgR and leucine for Lrp) and the A-R-A and A-R-R logical structures for other amino acids (FIG. 5c).

The regulons of ArgR, Lrp, and TrpR were constructed in E. coli individually and then integrated them to form the first genome-scale reconstruction of a stimulon. The TF-binding regions on the E. coli genome experimentally and furthermore to elucidate any DNA sequence motif(s) correlated with the TF regulatory action were established. Second, the size of each regulon was significantly extended and obtained 140, 283, and 15 target genes for each regulon. Third, using changes in transcript levels on a genome-scale, the regulatory modes for individual genes governed by each TF in response to exogenous arginine, leucine, and tryptophan were identified. The integrated analyses indicate that the functional assignment of the regulated genes is strongly enriched in amino acid metabolism-related functions. As suggested previously, many of these genes are likely to be involved in the “feast or famine” adaptation for survival in nutrient-rich or depleted environments. Fourth, the regulated target genes were assigned to three functional categories; transport, biosynthesis, and metabolism of amino acids. The classification allowed us to identify the connected circuit motif as a basic building block of the integrated network. Finally, the regulatory logic of the connected circuit motif based on the causal relationships between the association of TFs and changes in transcript levels was determined. These fall into two main categories and thus allow for the differentiation between amino acids as signaling and nutrient molecules.

In general, transport systems along with biosynthetic and metabolic pathways convert external resources to basic building blocks to sustain life. The coordinated regulation of this primary process underlies expression of optimized metabolic states under different external conditions. Thus, the logical structures of the metabolite-regulation connected circuit was examined in response to the changes in the external amino acid availability in the reconstructed stimulon. Three unique logical structures that govern the amino acid biosynthesis and metabolism were uncovered. The R-R-A logical structure was observed for signaling molecules whereas the A-R-A and A-R-R logical structures were determined for other amino acids serving as a nutrient source (FIG. 5a, b). In principle, every metabolic pathway that includes transport, biosynthesis, and utilization functions could follow these logical structures. For example, purine metabolism in E. coli contains a wide range of genes whose functions are transport (yieG), biosynthesis (cvpA-purF-ubiX, purHD, purMN, purT, purL, purEK, purC, hflD-purB, purA, and guaAB), utilization (apt), and a transcriptional regulator (purR). The metabolic functions of regulon members of PurR enriched in purine metabolism and the connected circuit motif indicated the logical structures for signaling molecules in response to exogenous purine. It can be therefore envisioned that other potential metabolic pathways follow similar logical structures as determined for amino acid metabolism in bacteria.

Bacterial cells import essential nutrients and inorganic ions such as galactose and iron due to the absence of the biosynthesis pathway. It is therefore of interest that the simple feedback circuit (SFL) motif, a connected circuit motif of transporter and utilization pathway by TF, is often observed in the regulatory circuits for these molecules. If it is assumed that the feedback circuit composed of influx and efflux combination, the logical structures of R-R-A, A-R-A, and A-R-R in the CFL motif can be reduced to R-A, A-A, and A-R, respectively. In E. coli, the galactose metabolic pathway is controlled by the galactose repressor (GalR) and galactose isorepressor (GalS), whereas iron homeostasis is controlled by the ferric uptake regulator (Fur). In the case of galactose metabolism, both GalR and GalS directly repress the transcription of galP encoding galactose permease. In a similar way, GalR partially represses the mglBAC operon encoding high-affinity, ABC-type transport system. When galactose is available in the medium, the DNA-binding by both GalR and GalS is inhibited, followed by the activation of those genes along with the genes for galactose utilization. Therefore, the SFL motif exhibits the A-A logical structure, confirming the exogenous galactose as nutrient. In the iron homeostasis system in E. coli, intracellular iron binds to Fur, forming the active TF complex, which in turn activates the production of iron-using metabolic enzymes and also shuts down expression of iron transporters. Interestingly, the SFL motif for Fur regulon exhibits the R-A logical structure, similar to amino acids serving as signaling molecules described above. Therefore, it can be concluded that iron acts as a signaling molecule rather than a nutrient.

The regulatory relationships between TFs and their target genes determine the regulatory logic of the TRN. TRNs are thought to contain a set of recurring regulatory motifs; such as single input module (SIM), feedforward loop (FFL), and dense overlapping regulons (DOR). The first motif, termed SIM is defined by a set of operons that are controlled by a single TF without additional transcriptional regulation input. It has been suggested that there are 24 systems that exhibit a SIM motif in E. coli. With the genome-scale elucidation of the regulons a comprehensively examine the existence of such regulatory motifs was conducted. The amino acid biosynthesis pathways such as arginine biosynthesis have been used as an example to demonstrate the existence of the SIM motif within the E. coli TRN. However, the genome-wide measurement of binding sites shows that ArgR regulates Lrp-L, which subsequently regulates the biosynthetic pathway for branched chain amino acids with autoregulation. In addition, Lrp regulates the transcription of the first enzyme (i.e., argA) in the arginine biosynthetic pathway (FIG. 3a). Therefore, the amino acid biosynthesis pathways are likely to belong to the DOR rather than the SIM pattern. Clearly, the genome-scale view now becoming available will lead to re-assessment of the regulatory logic deployed in operons, regulons and stimulons. Based on the hierarchical relationship between ArgR and Lrp-L (i.e., ArgR represses the transcription of Lrp-L), coherent and incoherent FFL motifs were observed from their regulons. For example, the two operons artMQIP and potFGHI are down-regulated by both ArgR and Lrp (i.e., incoherent FFL motif). On the other hand, the gltBD operon is down-regulated by ArgR but up-regulated by Lrp (i.e., coherent FFL motif). Based on the fact that ArgR directly regulates four, and Lrp twelve TFs, utilization of the FFL motif is a widely spread strategy to control the TRN in response to exogenous amino acids. With genome-scale data now becoming available, it would be expected that most of the regulatory motifs in the TRN will be DOR and FFL, and that the SIM motif might be less common than previously thought. Interestingly, exogenous leucine as input signal for the FFL motifs changes the logical structure of the FFL motif type due to the regulatory effect of leucine on the activity of Lrp-l. For instance, ArgR represses the artMQIP operon whereas Lrp-L induces its transcription in response to leucine. Therefore, the regulatory logic of FFL motifs varies with changes in the environmental condition demonstrating inherent network plasticity as a basic principle by which cells to adapt to changes.

Monitoring the exogenous nutritional state, the cell not only adjusts its metabolism to adapt the nutritional conditions in cooperation with the TRN but also change its genome structure by altering the binding patterns of nucleoid-associated proteins (NAP). The variation in transcript levels of the NAPs can thus provide a means to modulate the structure of the genome, depending on growth conditions. Interestingly, ArgR down-regulates the transcription of the NAPs dps and stpA in response to the exogenous arginine (FIG. 8). dps and stpA encode a DNA binding protein from starved cells and a H-NS-like DNA-binding protein with RNA chaperone activity, respectively. The transcription of stpA is up-regulated by Lrp, however the exogenous leucine reduces its transcript level through interfering the Lrp-binding to the promoter region. This regulatory effect results from the fact that the activity of Lrp can be potentiated, inhibited, or unaffected by leucine. Therefore, it is likely that external amino acids (at least leucine and arginine) act as signaling molecules to convey the environmental conditions to the cell. The nutrient level can therefore be an important cue for shaping the genome structure as well.

In summary, an integrative analysis of genome-scale data sets to comprehensively understand the basic principles governing a stimulon in the TRN of E. coli has been described. The overarching regulatory principle elucidated enabled us to differentiate between metabolites as signaling and nutrient molecules. This important distinction between seemingly similar metabolites is non-intuitive and could only be determined through genome-scale systems analysis. Similar analysis of other stimulons and large-scale regulatory networks may reveal that this regulatory principle is general. This approach to the analysis of regulation at the network level may reveal other fundamental and non-obvious regulatory principles at work in genome-scale regulatory networks.

Although the invention has been described with reference to the above example, it will be understood that modifications and variations are encompasses within the spirit and scope of the invention. Accordingly, the invention is limited only by the following claims.

1. A method of identifying a regulatory motif for amino acid metabolism in a target organism comprising:
(a) obtaining the full genome sequence a target organism;
(b) obtaining the genome-wide binding of a transcription factor from the organism;
(c) obtaining the sequence of the binding sites from the organism;
(d) obtaining the data described in (b) and (c) under a series of different culture conditions for the organism; and
(e) iteratively mapping the data sets described in (d) onto the DNA sequence in (a) and identify binding sites associated with genes involved in amino acid metabolism,
thereby identifying a regulatory motif for amino acid metabolism in the target organism.

2. The method of claim 1, wherein the target organism is a bacterial organism.

3. The method of claim 1, wherein the target organism is E. coli.

4. The method of claim 1, wherein the genome-wide binding of the transcription factor is obtained by chromatin immunoprecipitation coupled with a microarray.

5. The method of claim 1, wherein the genome-wide binding of the transcription factor is obtained by deep sequencing of immunoprecipitated DNA.

6. The method of claim 1, wherein the sequence of the binding sites is obtained using tiled expression arrays.

7. The method of claim 1, wherein the sequence of the binding sites is obtained using deep sequencing of the isolated DNA.

8. The method of claim 1, wherein the regulatory motif is associated with amino acid transport, biosynthesis or utilization.

9. The method of claim 1, wherein the transcription factor is selected from the group consisting of be ArgR, Lrp, TrpR, TyrR, PurR, PyrR, Fnr, ArcA, Crp, Cra, DgsA, Fis, Hns, HU, Ihf, StpA and Dps.

10. The method of claim 1, wherein one or more small molecules is used to produce the different culture conditions.

11. The method of claim 10, wherein the small molecule is an amino acid.

12. A regulatory motif for ArpR binding selected from the group consisting of SEQ ID NOs:1-126.

13. A regulatory motif for Lrp binding selected from the group consisting of SEQ ID NOs:127-265.

14. A regulatory motif for TrpR binding selected from the group consisting of SEQ ID NOs:266-279.

15. A method of modulating the activity of ArgR comprising contacting ArgR with a small molecule.

16. The method of claim 15, wherein the small molecule is an amino acid.

17. The method of claim 16, wherein the amino acid is selected from the group consisting of phenylalanine, tyrosine, tryptophan, lysine, arginine, histidine, aspartic acid and glutamic acid.

18. The method of claim 15, wherein the modulated activity is activation or repression of at least one pathway.

19. The method of claim 18, wherein the pathway is an amino acid transportation, biosynthesis or utilization pathway.

20. The method of claim 16 wherein the amino acid is phenylalanine, tyrosine or tryptophan and the modulated activity is activation of transportation or utilization pathway and repression of a biosynthesis pathway.

21. The method of claim 16 wherein the amino acid is lysine, arginine or histidine and the modulated activity is activation of a utilization pathway and repression of a biosynthesis and a transportation pathway.

22. The method of claim 16 wherein the amino acid is asparagine or glutamine and the modulated activity is activation of transportation pathway and repression of a utilization and a biosynthesis pathway.

23. A method of modulating the activity of Lrp comprising contacting Lrp with a small molecule.

24. The method of claim 23, wherein the small molecule is an amino acid.

25. The method of claim 24, wherein the amino acid is selected from the group consisting of phenylalanine, tyrosine, tryptophan, lysine, arginine, histidine, aspartic acid, glutamic acid, valine, isoleucine, leucine, alanine, glycine, serine, threonine and proline.

26. The method of claim 23, wherein the modulated activity is activation or repression of at least one pathway.

27. The method of claim 26, wherein the pathway is an amino acid transportation, biosynthesis or utilization pathway.

28. The method of claim 24, wherein the amino acid is phenylalanine, tyrosine or tryptophan and the modulated activity is activation of transportation or utilization pathway and repression of a biosynthesis pathway.

29. The method of claim 24, wherein the amino acid is lysine, arginine or histidine and the modulated activity is activation of a transportation or utilization pathway and repression of a biosynthesis pathway.

30. The method of claim 24, wherein the amino acid is asparagine or glutamic acid and the modulated activity is activation of a utilization or a biosynthesis pathway and repression of a transportation pathway.

31. The method of claim 24 wherein the amino acid is valine, isoleucine or leucine and the modulated activity is activation of a transportation or a biosynthesis pathway and repression of a utilization pathway.

32. The method of claim 24 wherein the amino acid is alanine, glycine, serine, threonine or proline and the modulated activity is activation of a transportation or a utilization or a pathway and repression of a biosynthesis pathway.
